(19) United States
(12) Patent Application Publication
(10) Pub. No.: US 2026/0183316 A1
(43) Pub. Date: Jul. 2, 2026

(54) CANNABICHROMENE-TYPE CANNABINOID COMPOSITIONS, METHODS AND USES THEREOF

(71) Applicant: Cronos Group Inc., Toronto (CA)

(72) Inventors: Aryeh ALTSHUL, Modiin (IL); Moran MAZUZ, Rishon Le-zion (IL); Aaron Arye WEIGENSBERG, Zichron Yaakov (IL); Shmuel GAVRIELOV, Jerusalem (IL); Tsahi SCHLINGER, Tel Aviv (IL); Ning BIAN, Toronto (CA); Pinchas Dovid STEINBERG, Chashmonaim (IL); Gordon Hagen, Tiny (CA); Claire Kerene LINTON, Nottawa (CA); Yanna-Nicol FREUDIGER, Tiny (CA); Robert Tait GALE, Toronto (CA)

(21) Appl. No.: 19/131,246

(22) PCT Filed: Nov. 15, 2023
(86) PCT No.: PCT/CA2023/051536

§ 371 Date: May 20, 2025

Related U.S. Application Data
(60) Provisional application No. 63/427,175, filed on Nov. 22, 2022.

(51) Int. Cl. A61K 31/00; A61K 9/00

(52) U.S. Cl. CPC A61K 31/658; A61K 9/0056

(57) ABSTRACT

Disclosed herein are compositions comprising a cannabichromene-type cannabinoid. More particularly, the present disclosure relates to compositions comprising a cannabichromene-type cannabinoid and a tetrahydrocannabinol-type cannabinoid.

### RELATED APPLICATION INFORMATION

This application claims priority to U.S. Application No. 63/427,175, filed on Nov. 16, 2022, the contents of which is herein incorporated by reference.

### FIELD

The present disclosure relates to compositions comprising a cannabichromene-type cannabinoid. More particularly, the present disclosure relates to compositions comprising a cannabichromene-type cannabinoid and a tetrahydrocannabinol-type cannabinoid. Even more particularly, the present disclosure relates to modulation of the tetrahydrocannabinol-type cannabinoids using cannabichromene-type cannabinoids.

### INTRODUCTION

Cannabis is a genus of flowering plants that has been used by humans for various purposes, such as medicines, ritual, recreation and textiles. The flowers of the cannabis plant include glandular trichomes, in which phytocannabinoids are produced.

Over 140 phytocannabinoids produced by the cannabis plant have been isolated and identified to date. The most well-known phytocannabinoids are tetrahydrocannabinolic acid (THCA) and cannabidiolic acid (CBDA). THCA, when decarboxylated, is transformed into tetrahydrocannabinol (THC). THC can also be prepared by chemical synthesis or plant extraction. THC is a substance that users may use in order to get a “high” when the cannabis flower is smoked or ingested orally. However, THC has also been shown to be useful for other purposes, such as an appetite stimulant for people with AIDS and an antiemetic for people undergoing chemotherapy. CBDA, when decarboxylated, is transformed into cannabidiol (CBD). CBD has been shown to be useful for treating certain types of epilepsy, and has other purported effects.

Other cannabinoids (sometimes called “minor cannabinoids”) typically are found in lower concentrations in the cannabis plant. Due to this lower prevalence, the minor cannabinoids have not been subject to the same level of research as THC and CBD. However, the minor cannabinoids have also been purported to have physiological, neurological, and/or therapeutic effects. In addition, a handful of pre-clinical studies have shown potential for some minor cannabinoids to modulate the effects of THC and/or CBD when co-administered.

Thus, there is a need for formulations that include minor cannabinoids.

### SUMMARY

In one embodiment, the present disclosure relates to a composition comprising a THC-type cannabinoid and a CBC-type cannabinoid. In some aspects, when the composition is administered to or consumed by a subject, it induces a modulated high in the subject as compared to a control composition that does not contain the CBC-type cannabinoid.

In other aspects, the modulated high in the subject includes a modulated impairment, mood alteration, a combination thereof, as compared to the control composition. In still yet further aspects, the modulated impairment in the subject includes a modulated:
impairment duration;
sensory perception;
executive function or cognition;
memory; or
any combination thereof,
as compared to a control composition that does not contain a CBC-type cannabinoid.

Additionally, in yet further aspects, the modulated mood alteration in the subject includes a modulated:
mood alteration duration;
sociability;
anger;
confusion;
depression;
fatigue;
despair;
tension;
vigor;
grit;
resiliency;
sociability;
willingness to explore; or
any combination thereof,
as compared to a control composition without the CBC-type cannabinoid.

In still other aspects, the composition further comprises a CBC-type cannabinoid:THC-type cannabinoid ratio (CBC:THC) of from 1:1 to 100:1. In still other aspects, the composition further comprises a CBC-type cannabinoid:THC-type cannabinoid ratio (CBC:THC) of from 100:1 to 1:100 with the proviso that when the cannabinoid:THC-type cannabinoid (CBC:THC) ratio is 1.1:0.9 and cannabidiol (CBD) is present in the composition, the ratio of CBD:CBC:THC is not 20:1.1:0.9 (CBD:CBC:THC). In still other aspects, the composition further comprises a CBC-type cannabinoid:THC-type cannabinoid ratio (CBC:THC) of from 20:1 to 1:20. In still yet further aspects, the CBC-type cannabinoid:THC-type cannabinoid ratio (CBC:THC) is from 15:1 to 1:15. In still yet further aspects, the CBC-type cannabinoid:THC-type cannabinoid ratio (CBC:THC) is from 10:1 to 1:10. In still yet further aspects, the CBC-type cannabinoid:THC-type cannabinoid ratio is from 6:1 to 1:9. In still yet further aspects, the CBC-type cannabinoid:THC-type cannabinoid ratio is from 5:1 to 1:8. In still yet further aspects, the CBC-type cannabinoid:THC-type cannabinoid ratio (CBC:THC) is from 4:1 to 1:7.

In yet still other aspects, the CBC-type cannabinoid: THC-type cannabinoid ratio (CBC:THC) is about 20:1. In yet still other aspects, the CBC-type cannabinoid: THC-type cannabinoid ratio (CBC:THC) is about 19:1. In yet still other aspects, the CBC-type cannabinoid: THC-type cannabinoid ratio (CBC:THC) is about 18:1. In yet still other aspects, the CBC-type cannabinoid: THC-type cannabinoid ratio (CBC:THC) is about 17:1. In yet still other aspects, the CBC-type cannabinoid: THC-type cannabinoid ratio (CBC:THC) is about 16:1. In yet still other aspects, the CBC-type cannabinoid: THC-type cannabinoid ratio (CBC:THC) is about 15:1. In yet still other aspects, the CBC-type cannabinoid: THC-type cannabinoid ratio (CBC:THC) is about 14:1. In yet still other aspects, the CBC-type cannabinoid: THC-type cannabinoid ratio (CBC:THC) is about 13:1. In yet still other aspects, the CBC-type cannabinoid: THC-type cannabinoid ratio (CBC:THC) is about 12:1. In yet still other aspects, the CBC-type cannabinoid: THC-type cannabinoid ratio (CBC:THC) is about 11:1. In yet still other aspects, the CBC-type cannabinoid: THC-type cannabinoid ratio (CBC:THC) is about 10:1. In yet still other aspects, the CBC-type cannabinoid: THC-type cannabinoid ratio (CBC:THC) is about 9:1. In yet still other aspects, the CBC-type cannabinoid: THC-type cannabinoid ratio (CBC:THC) is about 8:1. In yet still other aspects, the CBC-type cannabinoid: THC-type cannabinoid ratio (CBC:THC) is about 7:1. In yet still other aspects, the CBC-type cannabinoid: THC-type cannabinoid ratio (CBC:THC) is about 6:1. In yet still other aspects, the CBC-type cannabinoid: THC-type cannabinoid ratio (CBC:THC) is about 5:1. In yet still other aspects, the CBC-type cannabinoid: THC-type cannabinoid ratio (CBC:THC) is about 4:1. In yet still other aspects, the CBC-type cannabinoid: THC-type cannabinoid ratio (CBC:THC) is about 3:1. In yet still other aspects, the CBC-type cannabinoid: THC-type cannabinoid ratio (CBC:THC) is about 2:1. In still yet other aspects, the CBC-type cannabinoid:THC-type cannabinoid ratio (CBC:THC) is about 1:2. In still yet other aspects, the CBC-type cannabinoid:THC-type cannabinoid ratio (CBC:THC) is about 1:3. In still yet other aspects, the CBC-type cannabinoid:THC-type cannabinoid ratio (CBC:THC) is about 1:4. In still yet other aspects, the CBC-type cannabinoid:THC-type cannabinoid ratio (CBC:THC) is about 1:5. In still yet other aspects, the CBC-type cannabinoid:THC-type cannabinoid ratio (CBC:THC) is about 1:6. In still yet other aspects, the CBC-type cannabinoid:THC-type cannabinoid ratio (CBC:THC) is about 1:7 In still yet other aspects, the CBC-type cannabinoid:THC-type cannabinoid ratio (CBC:THC) is about 1:8. In still yet other aspects, the CBC-type cannabinoid:THC-type cannabinoid ratio (CBC:THC) is about 1:9. In still yet other aspects, the CBC-type cannabinoid:THC-type cannabinoid ratio (CBC:THC) is about 1:10. In still yet other aspects, the CBC-type cannabinoid:THC-type cannabinoid ratio (CBC:THC) is about 1:11. In still yet other aspects, the CBC-type cannabinoid:THC-type cannabinoid ratio (CBC:THC) is about 1:12. In still yet other aspects, the CBC-type cannabinoid:THC-type cannabinoid ratio (CBC:THC) is about 1:13. In still yet other aspects, the CBC-type cannabinoid:THC-type cannabinoid ratio (CBC:THC) is about 1:14. In still yet other aspects, the CBC-type cannabinoid:THC-type cannabinoid ratio (CBC:THC) is about 1:15. In still yet other aspects, the CBC-type cannabinoid:THC-type cannabinoid ratio (CBC:THC) is about 1:16. In still yet other aspects, the CBC-type cannabinoid:THC-type cannabinoid ratio (CBC:THC) is about 1:17. In still yet other aspects, the CBC-type cannabinoid:THC-type cannabinoid ratio (CBC:THC) is about 1:18. In still yet other aspects, the CBC-type cannabinoid:THC-type cannabinoid ratio (CBC:THC) is about 1:19. In still yet other aspects, the CBC-type cannabinoid:THC-type cannabinoid ratio (CBC:THC) is about 1:20.

In still further aspects, the composition comprises from about 4% to about 99% of the CBC-type cannabinoid and from about 1% to about 96% of the THC-type cannabinoid by weight of total cannabinoids.

In yet further aspects, the composition comprises from about 0.02 mg to about 5000 mg of the CBC-type cannabinoid and from about 0.5 mg to about 5000 mg by weight of the THC-type cannabinoid.

In still further aspects, the composition further comprises cannabicyclol (CBL), cannabicitran (CBT), sesqui-CBC, sesqui-CBG, cannabidiol (CBD), cannabigerol (CBG), or any combinations thereof.

In yet still further aspects, the THC-type cannabinoid in the composition comprises tetrahydrocannabinol (THC), tetrahydrocannabivarinol (THCV), tetrahydrocannabibutol (THCB), tetrahydrocannabihexol (THCH), tetrahydrocannabiphorol (THCP), hexahydrocannabinol (HHC), hexahydrocannabivarinol (HHCV), hexahydrocannabibutol (HHCB), hexahydrocannabihexol (HHCH), hexahydrocannabiphorol (HHCP), cannabinol (CBN), cannabivarinol (CBNV), cannabibutol (CBNB), cannabihexol (CBNH), cannabiphorol (CBNP), tetrahydrocannabinol-o-acetate (THC-O-Ac), tetrahydrocannabivarinol-o-acetate (THCV-O-Ac), tetrahydrocannabibutol-o-acetate (THCB-O-Ac), tetrahydrocannabihexol-o-acetate (THCH-O-Ac), tetrahydrocannabiphorol-o-acetate (THCP-O-Ac), 11-hydroxy-tetrahydrocannabinol (11-OH-THC), 11-hydroxy-tetrahydrocannabivarinol (11-OH-THCV), 11-hydroxy-tetrahydrocannabibutol (11-OH-THCB), 11-hydroxy-tetrahydrocannabihexol (11-OH-THCH), 11-hydroxy-tetrahydrocannabiphorol (11-OH-THCP), or a combination thereof. In still further aspects, the THC-type cannabinoid comprises THC. In yet other aspects, the THC comprises delta-8 THC, delta-9 THC, or both. In still further aspects, the THC comprises delta-9 THC.

In still further aspects, the CBC-type cannabinoid comprises cannabichromevarin (CBCV), cannabichromebutol (CBCB), cannabichromene (CBC), cannabichromehexol (CBCH), cannabichromephorol (CBCP), cannabichromebutol (CBCB), cannabichromehexol (CBCH), sesqui-CBC, sesqui-CBCV, sesqui-CBCB, sesqui-CBCH, sesqui-CBCP, or a combination thereof. More specifically, in yet other aspects, the CBC-type cannabinoid comprises CBC.

In still further aspects, the composition is for oral or buccal administration or oral or buccal consumption. When the composition is for oral or buccal administration, or oral or buccal consumption, the composition further comprises a taste masking agent.

In still further aspects, the composition is for pulmonary administration.

In still further aspects, the composition is a dosage form. For example, in some aspects, the dosage form is a tablet, a liquid, a capsule, a film, a powder, an aerosol, a gel, a cream, an ointment, or any combination thereof. In yet further aspects, the dosage form further comprises at least one pharmaceutically acceptable carrier. In still further aspects, when the dosage form is a tablet, liquid capsule, or film, the dosage form further comprises a masking agent.

In another embodiment, the above composition is used to deliver a modulated effect of a THC-type cannabinoid.

In yet another embodiment, the present disclosure relates to a product comprising the above described composition. In some aspects, the product comprises an inhalable cannabis product; an ingestible cannabis product; a topical cannabis product or any combination thereof. In yet other aspects, the product comprises an inhalable cannabis product, an ingestible cannabis product, or any combination thereof.

In some aspects, the product comprises an inhalable or ingestible cannabis product.

In still other aspects, the product is a vaporizer, vaporizer cartridge, vaporizer composition, joint, blunt, or spliff.

In still further aspects, the product is a cannabis edible, beverage, or a tincture.

In yet further aspects, the cannabis edible is a gummy, chocolate, confection or baked good. More specifically, in some aspects, the cannabis edible is a gummy. In other aspects, the cannabis edible is chocolate. In yet other aspects, the cannabis edible is a confection. In still further aspects, the cannabis edible is a baked good.

In yet another embodiment, the present disclosure relates to a method of delivering a modulated effect of a THC-type cannabinoid comprising administering to a subject the above composition. In yet other embodiments, the present disclosure relates to a method of delivering a modulated effect of a THC-type cannabinoid to a subject, wherein the method comprises having the subject consume a THC-type cannabinoid to provide the modulated effect.

In another embodiment, the present disclosure relates to a composition for use with a THC-type cannabinoid, wherein the composition comprises a CBC-type cannabinoid. In some aspects, when the THC-type cannabinoid and composition are administered to a subject, the THC-type cannabinoid induces a high in the subject, and the composition modulates the high. In yet further aspects, the high includes impairment, mood alteration, or both in the subject, and the composition modulates the impairment, mood alteration, or both in the subject. In yet further aspects, the modulation of the impairment of the subject comprises modulation of:
impairment duration;
sensory perception;
executive function (or cognition);
memory; or
any combination thereof.

In still further aspects, the modulation of the mood alteration of the subject comprises modulation of:
mood alteration duration;
sociability;
anger;
confusion;
depression;
fatigue;
despair;
tension;
vigor;
grit;
resiliency;
sociability; or
any combination thereof.

In still other aspects of the above composition, the CBC-type cannabinoid is adapted to be included with the THC-type cannabinoid in a CBC-type cannabinoid:THC-type cannabinoid ratio (CBC:THC) of from 100:1 to 1:100. In still other aspects, the composition further comprises a CBC-type cannabinoid:THC-type cannabinoid ratio (CBC:THC) of from 100:1 to 1:100 with the proviso that when the cannabinoid:THC-type cannabinoid (CBC:THC) ratio is 1.1:0.9 and cannabidiol (CBD) is present in the composition, the ratio of CBD:CBC:THC is not 20:1.1:0.9 (CBD:CBC:THC). In still other aspects, the composition further comprises a CBC-type cannabinoid:THC-type cannabinoid ratio (CBC:THC) of from 20:1 to 1:20. In still yet further aspects, the CBC-type cannabinoid:THC-type cannabinoid ratio (CBC:THC) is from 15:1 to 1:15. In still yet further aspects, the CBC-type cannabinoid:THC-type cannabinoid ratio (CBC:THC) is from 10:1 to 1:10. In still yet further aspects, the CBC-type cannabinoid:THC-type cannabinoid ratio is from 6:1 to 1:9. In still yet further aspects, the CBC-type cannabinoid:THC-type cannabinoid ratio is from 5:1 to 1:8. In still yet further aspects, the CBC-type cannabinoid:THC-type cannabinoid ratio (CBC:THC) is from 4:1 to 1:7.

In yet still other aspects, the CBC-type cannabinoid: THC-type cannabinoid ratio (CBC:THC) is about 20:1. In yet still other aspects, the CBC-type cannabinoid: THC-type cannabinoid ratio (CBC:THC) is about 19:1. In yet still other aspects, the CBC-type cannabinoid: THC-type cannabinoid ratio (CBC:THC) is about 18:1. In yet still other aspects, the CBC-type cannabinoid: THC-type cannabinoid ratio (CBC:THC) is about 17:1. In yet still other aspects, the CBC-type cannabinoid: THC-type cannabinoid ratio (CBC:THC) is about 16:1. In yet still other aspects, the CBC-type cannabinoid: THC-type cannabinoid ratio (CBC:THC) is about 15:1. In yet still other aspects, the CBC-type cannabinoid: THC-type cannabinoid ratio (CBC:THC) is about 14:1. In yet still other aspects, the CBC-type cannabinoid: THC-type cannabinoid ratio (CBC:THC) is about 13:1. In yet still other aspects, the CBC-type cannabinoid: THC-type cannabinoid ratio (CBC:THC) is about 12:1. In yet still other aspects, the CBC-type cannabinoid: THC-type cannabinoid ratio (CBC:THC) is about 11:1. In yet still other aspects, the CBC-type cannabinoid: THC-type cannabinoid ratio (CBC:THC) is about 10:1. In yet still other aspects, the CBC-type cannabinoid: THC-type cannabinoid ratio (CBC:THC) is about 9:1. In yet still other aspects, the CBC-type cannabinoid: THC-type cannabinoid ratio (CBC:THC) is about 8:1. In yet still other aspects, the CBC-type cannabinoid: THC-type cannabinoid ratio (CBC:THC) is about 7:1. In yet still other aspects, the CBC-type cannabinoid: THC-type cannabinoid ratio (CBC:THC) is about 6:1. In yet still other aspects, the CBC-type cannabinoid: THC-type cannabinoid ratio (CBC:THC) is about 5:1. In yet still other aspects, the CBC-type cannabinoid: THC-type cannabinoid ratio (CBC:THC) is about 4:1. In yet still other aspects, the CBC-type cannabinoid:THC-type cannabinoid ratio (CBC:THC) is about 3:1. In yet still other aspects, the CBC-type cannabinoid: THC-type cannabinoid ratio (CBC:THC) is about 2:1. In still yet other aspects, the CBC-type cannabinoid:THC-type cannabinoid ratio (CBC:THC) is about 1:2. In still yet other aspects, the CBC-type cannabinoid:THC-type cannabinoid ratio (CBC:THC) is about 1:3. In still yet other aspects, the CBC-type cannabinoid:THC-type cannabinoid ratio (CBC:THC) is about 1:4. In still yet other aspects, the CBC-type cannabinoid:THC-type cannabinoid ratio (CBC:THC) is about 1:5. In still yet other aspects, the CBC-type cannabinoid:THC-type cannabinoid ratio (CBC:THC) is about 1:6. In still yet other aspects, the CBC-type cannabinoid:THC-type cannabinoid ratio (CBC:THC) is about 1:7 In still yet other aspects, the CBC-type cannabinoid:THC-type cannabinoid ratio (CBC:THC) is about 1:8. In still yet other aspects, the CBC-type cannabinoid:THC-type cannabinoid ratio (CBC:THC) is about 1:9. In still yet other aspects, the CBC-type cannabinoid:THC-type cannabinoid ratio (CBC:THC) is about 1:10. In still yet other aspects, the CBC-type cannabinoid:THC-type cannabinoid ratio (CBC:THC) is about 1:11. In still yet other aspects, the CBC-type cannabinoid:THC-type cannabinoid ratio (CBC:THC) is about 1:12. In still yet other aspects, the CBC-type cannabinoid:THC-type cannabinoid ratio (CBC:THC) is about 1:13. In still yet other aspects, the CBC-type cannabinoid:THC-type cannabinoid ratio (CBC:THC) is about 1:14. In still yet other aspects, the CBC-type cannabinoid:THC-type cannabinoid ratio (CBC:THC) is about 1:15. In still yet other aspects, the CBC-type cannabinoid:THC-type cannabinoid ratio (CBC:THC) is about 1:16. In still yet other aspects, the CBC-type cannabinoid:THC-type cannabinoid ratio (CBC:THC) is about 1:17. In still yet other aspects, the CBC-type cannabinoid:THC-type cannabinoid ratio (CBC:THC) is about 1:18. In still yet other aspects, the CBC-type cannabinoid:THC-type cannabinoid ratio (CBC:THC) is about 1:19. In still yet other aspects, the CBC-type cannabinoid:THC-type cannabinoid ratio (CBC:THC) is about 1:20.

In still further aspects, the composition further comprises cannabicyclol (CBL), cannabicitran (CBT), sesqui-CBC, sesqui-CBG, cannabidiol (CBD), cannabigerol (CBG), or any combinations thereof.

In still other aspects, the THC-type cannabinoid in the composition comprises tetrahydrocannabinol (THC), tetrahydrocannabivarinol (THCV), tetrahydrocannabibutol (THCB), tetrahydrocannabihexol (THCH), tetrahydrocannabiphorol (THCP), hexahydrocannabinol (HHC), hexahydrocannabivarinol (HHCV), hexahydrocannabibutol (HHCB), hexahydrocannabihexol (HHCH), hexahydrocannabiphorol (HHCP), cannabinol (CBN), cannabivarinol (CBNV), cannabibutol (CBNB), cannabihexol (CBNH), cannabiphorol (CBNP), tetrahydrocannabinol-o-acetate (THC-O-Ac), tetrahydrocannabivarinol-o-acetate (THCV-O-Ac), tetrahydrocannabibutol-o-acetate (THCB-O-Ac), tetrahydrocannabihexol-o-acetate (THCH-O-Ac), tetrahydrocannabiphorol-o-acetate (THCP-O-Ac), 11-hydroxy-tetrahydrocannabinol (11-OH-THC), 11-hydroxy-tetrahydrocannabivarinol (11-OH-THCV), 11-hydroxy-tetrahydrocannabibutol (11-OH-THCB), 11-hydroxy-tetrahydrocannabihexol (11-OH-THCH), 11-hydroxy-tetrahydrocannabiphorol (11-OH-THCP), or any combinations thereof. In still further aspects, the THC-type cannabinoid comprises THC. In still yet other aspects, the THC comprises delta-8 THC, delta-9 THC, or both. In still further aspects, the THC comprises delta-9 THC.

In yet other aspects of the composition, the CBC-type cannabinoid comprises cannabichromevarin (CBCV), cannabichromebutol (CBCB), cannabichromene (CBC), cannabichromehexol (CBCH), cannabichromephorol (CBCP), cannabichromebutol (CBCB), cannabichromehexol (CBCH), sesqui-CBC, sesqui-CBCV, sesqui-CBCB, sesqui-CBCH, sesqui-CBCP, or any combination thereof. In some aspects, the CBC-type cannabinoid comprises CBC.

In yet further aspects, the composition is used for oral, buccal, pulmonary administration, or any combinations thereof.

In yet further aspects, the above composition is used to modulate the effects of a THC-type cannabinoid.

In yet another embodiment, the present disclosure relates to a method of modulating the effect of a THC-type cannabinoid to be administered to a subject comprising administered a THC-type cannabinoid to the subject, and administering the above composition to the subject, wherein the THC-type cannabinoid and the CBC-type cannabinoid can be administered separately to the subject or in a single composition.

### BRIEF DESCRIPTION OF THE FIGURES

FIG. 1 shows cannabinoid modulation of catalepsy, body temperature, and nociception in C57BL/6 mice. Male and female mice were treated with 0 (vehicle, olive oil) or 0.3-30 mg/kg p.o. compounds. For 1:6.92 (CBC:THC) and 3.90:1 (CBC:THC) products, this dosing was based on the mg/kg dose of THC received. Physiological assessments of (FIG. 1A, FIG. 1BE) catalepsy (5 min, % MPE 60 sec), (FIG. 1C,1D) body temperature (10 min), and (FIG. 1E,1F) nociception in the tail flick assay (52° C., 20 min % MPE 20 sec) were made. Data are mean±S.E.M. Panels on the left show all individual data points. Panels on the right show the same data fit to a log [concentration] vs. response 3-parameter model in GraphPad Prism (v. 9.2). N=6-8 mice/treatment.

FIG. 2 shows cannabinoid modulation of movement in the OFT in C57BL/6 mice. Male and female mice were treated with 0 (vehicle, olive oil) or 0.3-30 mg/kg p.o. compounds. For 1:6.92 (CBC:THC) and 3.90:1 (CBC:THC) products, this dosing was based on the mg/kg dose of THC received. Locomotion (FIG. 2A, 2B) and time in the central quadrant of the OFT (FIG. 2C, 2D) were made 30 min after compound administration. Data are mean±S.E.M. Panels on the left show all individual data points. Panels on the right show the same data fit to a log [concentration] vs. response 3-parameter model in GraphPad Prism (v. 9.2). N=6-8 mice/treatment.

FIG. 3 shows cannabinoid modulation of movement in the EPM in C57BL/6 mice. Male and female mice were treated with 0 (vehicle, olive oil) or 10 mg/kg p.o. compounds. For 1:6.92 (CBC:THC) and 3.90:1 (CBC:THC) products, this dosing was based on the mg/kg dose of THC received. Locomotion (FIG. 3A), open arm entries (FIG. 3B), closed arm entries (FIG. 3C), total arm entries (FIG. 3D), time spent in the open (FIG. 3E) and closed arms (FIG. 3F) and in the central quadrant of the EPM (FIG. 3G) were made 30 min after compound administration. Data are mean±S.E.M. with individual data points. N=6-7 mice/treatment.

FIG. 4 shows cannabinoid modulation of behaviour in the y-maze in C57BL/6 mice. Male and female mice were treated with 0 (vehicle, olive oil) or 10 mg/kg p.o. compounds. For 1:6.92 (CBC:THC) and 3.90:1 (CBC:THC) products, this dosing was based on the mg/kg dose of THC received. Locomotion (FIG. 4A), total arm entries (FIG. 4B), and % correct alternations (FIG. 4C) and in y-maze were made 40 min after compound administration. Data are mean±S.E.M. with individual data points. N=4-6 mice/treatment.

FIG. 5 shows cannabinoid modulation of behaviour in the FST in C57BL/6 mice. Male and female mice were treated with 0 (vehicle, olive oil) or 10 mg/kg p.o. compounds. For 1:6.92 (CBC:THC) and 3.90:1 (CBC:THC) products, this dosing was based on the mg/kg dose of THC received. Time swimming (FIG. 5A) and time immobile (FIG. 5B) were measured 50 min after compound administration. Data are mean±S.E.M. with individual data points. N=6-10 mice/treatment.

FIG. 6 shows pharmacokinetic measurement of CBC and THC in C57BL/6 mice. Male and female mice were treated with 10 mg/kg i.v. or p.o. of compounds. For 1:6.92 (CBC:THC) and 3.90:1 (CBC:THC) products, this dosing was based on the mg/kg dose of THC received. Blood samples were drawn 10 min, 1 h, 6 h, and 12 h after compound administration. Data are mean±S.E.M. with individual data points. N=3-6 mice/treatment.

FIG. 7 shows pharmacokinetic measurement of 11-OH-THC and COOH-THC in C57BL/6 mice. Male and female mice were treated with 10 mg/kg i.v. or p.o. of compounds. For 1:6.92 (CBC:THC) and 3.90:1 (CBC:THC) products, this dosing was based on the mg/kg dose of THC received. Blood samples were drawn 10 min, 1 h, 6 h, and 12 h after compound administration. Data are mean±S.E.M. with individual data points. N=3-6 mice/treatment.

### DETAILED DESCRIPTION

The present disclosure now will be described more fully with reference to the drawings, in which some, but not all embodiments of the disclosure are shown. The description may be embodied in many different forms and should not be construed as being limited to the embodiments set forth herein. Like numbers refer to like elements throughout.

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, specific examples of appropriate materials and methods are described herein.

Throughout the present specification and the accompanying claims the words “comprise,” “include,” and “have” and variations thereof such as “comprises,” “comprising,” “includes,” “including,” “has,” and “having” are to be interpreted inclusively. That is, these words are intended to convey the possible inclusion of other elements or integers not specifically recited, where the context allows. No language in the specification should be construed as indicating any non-claimed element essential to the practice of the disclosure.

As used herein, the singular forms “a”, “an”, and “the” refer to both the singular as well as plural, unless the context clearly indicates otherwise. For example, the term “a flower” includes single or plural flowers and can be considered equivalent to the phrase “at least one flower”.

The dimensions and values disclosed herein should not be understood as being strictly limited to the exact numerical values recited. Rather, unless otherwise specified, each dimension or value is intended to mean both the recited value and a functionally equivalent range surrounding that value as interpreted by those skilled in the art.

The expression “up to” includes amounts of zero to the expressed upper limit and all values therebetween. When ranges are specified, the range includes all values therebetween such as in increments of 0.1.

As used herein, the word “about”, should be given an interpretation that would be understood by a person of skill in the art. For example, in some circumstances, the associated numerical value includes a tolerance of ±10% around the stated numerical value. In some circumstances when used in connection with a numerical value that includes a recited range of percentages, the numerical value includes a tolerance of ±0.5% on the upper and lower limits of the range.

When reference is made to percentages in this specification, unless otherwise stated, it is intended that those percentages are based on weight, i.e., weight percentages.

Headings provided in this disclosure are for ease of reference and are not intended to limit the scope of the disclosure or claims.

As used herein, the term “material”, as it relates to chemical compounds, refers to a composition that includes one or more distinct compounds, and includes both pure substances and mixtures of different compounds. For example, a cannabinoid material will comprise one or more distinct cannabinoid molecules, but may also include other compounds such as terpenes. In another example, a cannabis material is a material that includes cannabis or a derivative thereof, such as cannabis extract, cannabis distillate, and isolated cannabinoids.

As used herein, the term “cannabis” means a plant of genus Cannabis, and unless the context clearly indicates otherwise, includes any part of the plant, such as the stalks, branches, leaves, flowers, roots, seed, and any combinations thereof. Cannabis is an annual, dioecious, flowering herb. Cannabis flowers contain trichomes, which are structures where certain compounds, including phytocannabinoids and terpenes, are secreted. Various taxonomical structures of plants of genus Cannabis have been proposed, such as those including a single species, Cannabis sativa, or as multiple species that additionally includes Cannabis indica and/or Cannabis ruderalis, which are considered subspecies under the single species classification. Additionally, cannabis includes hemp, which is generally understood to mean cannabis plants with low THC content.

As used herein, the term “cannabinoid” means a prenylated resorcinol, a prenylated beta-resorcylic acid, or a salt, ether derivative, ester derivative, hydrogenated derivative, or hydroxylated derivate thereof. Unless the context indicates otherwise, includes cannabinoids produced by plants, chemical synthesis and biosynthesis. In some embodiments, the prenylated resorcinols and prenylated beta-resorcylic acids have the structure of Formula I:

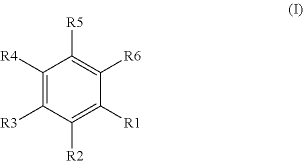

wherein:
R¹ is a hydrogen, an optionally substituted C₁-C₁₁ alkyl, an optionally substituted C₁-C₁₁ alkenyl, an optionally substituted C₁-C₁₁ alkynyl, or an optionally substituted C₁-C₁₁ aralkyl;
R² and R⁶ are, independently, hydrogen or carboxyl;
R³ and R⁵ are, independently, hydroxyl, C1-C₃ alkoxyl, acyl-substituted hydroxyl, or halogen; and
R⁴ is an optionally substituted prenyl moiety;
wherein R⁴ optionally cyclizes to itself, R³, R⁵, or a combination thereof.

In some embodiments, R¹ is a C₃-C₇ alkyl. In some embodiments, R¹ is a propyl, pentyl, or heptyl. In some embodiments, R¹ is propyl or pentyl. In some embodiments, R¹ is pentyl.

In some embodiments, R² is hydrogen.

In some embodiments, R³ and R⁵ are hydroxyl. In some embodiments, one of R³ and R⁵ is hydroxyl and the other is a methoxy or acetyl ester. In some embodiments, one of R³ and R⁵ is hydroxyl and the other is acetyl ester.

In some embodiments, R⁴ is geranyl, neryl, or farnesyl. In some embodiments, R⁴ is geranyl. In some embodiments, R⁴ is a geranyl cyclized to form a (1R,2R)-5-methyl-2-(prop-1-en-2-yl)-1,2,3,4-tetrahydro-1,1′-biphenyl moiety. In some embodiments, R⁴ is a geranyl cyclized to R³ to form a (6aR,10aR)-6,6,9-trimethyl-6a,7,8,10a-tetrahydro-6H-benzo[c]chromene moiety. In some embodiments, R⁴ is geranyl and cycliced to R³ to form a 2-methyl-2-(4-methylpent-3-en-1-yl)-7-pentyl-2H-chromene moiety.

Cannabinoids are often classified by “type,” i.e., by the topological arrangement of their prenyl moieties (See, for example, M. A. Elsohly and D. Slade, Life Sci., 2005, 78, 539-548; and L. O. Hanus et al. Nat. Prod. Rep., 2016, 33, 1357, which are herein incorporated by reference).

Generally, each “type” of cannabinoid includes the variations possible for ring substitutions of the resorcinol moiety at the position meta to the two hydroxyl moieties (the “tail”). In some embodiments, the tail for each “type” is an optionally substituted C₁-C₁₁ alkyl, an optionally substituted C₁-C₁₁ alkenyl, an optionally substituted C₁-C₁₁ alkynyl, or an optionally substituted C₁-C₁₁ aralkyl. The most common tail of a phytocannabinoid is a pentyl tail. Other common phytocannabinoid tails include propyl, heptyl, and butyl. Additional cannabinoid tails have been observed in other organisms. For example, methyl tails have been observed in daurichromenic acid and grifolic acid, and 2-benzylethyl has been observed in perrottetinene. Cannabinoids with other tails have been synthesized in labs (See, for example, Luo, X., Reiter, M. A., d'Espaux, L., Wong, J., Denby, C.M., Lechner, A., Zhang, Y., Grzybowski, A.T., Harth, S., Lin, W. and Lee, H., 2019. Complete biosynthesis of cannabinoids and their unnatural analogues in yeast. Nature, 567(7746), pp. 123-126).

Unless otherwise specified, a “type” of cannabinoid also includes derivatives thereof, including decarboxylated, hydrogenated, ethers, and esterified forms.

As used herein, a “CBG-type” cannabinoid refers to compounds having a 3-[(2E)-3,7-dimethylocta-2,6-dienyl]-2,4-dihydroxybenzoic acid moiety optionally substituted at the 6 position of the benzoic acid moiety, and is optionally decarboxylated.

As used herein, “CBC-type” cannabinoids refer to 5-hydroxy-2-methyl-2-(4-methylpent-3-enyl)-chromene-6-carboxylic acid optionally substituted at the 7-position of the chromene moiety, and is optionally decarboxylated.

As used herein, a “THC-type” cannabinoid is a (6aR,10aR)-1-hydroxy-6,6,9-trimethyl-6a,7,8,10a-tetrahydrobenzo[c]chromene-2-carboxylic acid optionally substituted at the 3-position of the benzo[c]chromene moiety, is optionally decarboxylated, and includes isomers where the double bond on the cyclohexene moiety of the tetrahydrobenzo[c]chromene are at different positions. For example, tetrahydrocannabinol includes all isomers having a double bond located at different positions on the cyclohexene moiety. As used herein, tetrahydrocannabinol or “THC”, without reference to the position of the double bond on the cyclohexene moiety, is intended include any such isomers, including Δ^{6a(7)}-THC, Δ⁷-THC, Δ⁸-THC, Δ⁹-THC, Δ¹⁰-THC, Δ^{6a(10a)}-THC. Similarly, tetrahydrocannabivarin or “THCV” includes all isomers where the double bond is at different positions on the cyclohexene moiety, and tetrahydrocannabiphorol or “THCP” includes all isomers where the double bond is at different positions on the cyclohexene moiety.

As used herein, a “CBD-type” cannabinoid is a 2,4-dihydroxy-3-[(1 R,6R)-3-methyl-6-prop-1-en-2-ylcyclohex-2-en-1-yl]-benzoic acid optionally substituted at the 6-position of the benzoic acid moiety, and is optionally decarboxylated.

In some embodiments, cannabinoid includes cannabigerol (“CBG”; CAS #25654-31-3); Δ⁹-tetrahydrocannabinol (“Δ⁹-THC”; CAS #1972-08-3); Δ⁸-tetrahydrocannabinolic acid (“Δ⁸-TH”; CAS #5957-75-5); cannabidiol (“CBD”; CAS #13956-29-1); cannabichromene (“CBC”; CAS #20675-51-8); cannabigerovarin (“CBGV”; CAS #55824-11-8); Δ⁹-tetrahydrocannabivarin (“Δ⁹-THCV”; CAS #31262-37-0); As-tetrahydrocannabivarin (“Δ⁸-THCV”; CAS #31262-38-1); cannabidivarin (“CBDV”; CAS #24274-48-4); cannabichromevarin (“CBCV”; CAS #41408-19-9); cannabinol (“CBN”, CAS #521-35-7); acidic forms of the foregoing and salts of the acidic forms.

As used herein, the term “phytocannabinoid” means a cannabinoid that is naturally produced by cannabis plants, and includes the acidic and decarboxylated acid forms of the naturally-occurring cannabis plant-derived cannabinoids and their degradants formed through storage or conventional use (e.g. by combustion, in bhang, etc.), such as through heat, UV or oxidative means, and also cannabinoids produced from synthetic and biosynthetic methods that are identical to naturally-occurring plant-derived cannabinoids. See, for example, Carvalho, A., Hansen, E.H., Kayser, O., Carlsen, S. and Stehle, F., 2017. Designing microorganisms for heterologous biosynthesis of cannabinoids. FEMS Yeast Research, 17(4).

As used herein, the term “modulate(s)” or “modulating” means upregulating or down regulating a response or activity, such as, for example, enhancing (e.g., increasing) or inhibiting (e.g., decreasing) a response or activity. By way of example, in the context of the present disclosure, “modulate” or “modulating” can refer to an increase or decrease in a character of the high experienced by a subject who is administered or consumes one or more compositions of the present disclosure when compared to a subject who is not administered or does not consume one or more compositions of the present disclosure (e.g., a control).

As used herein, “mood” means a set of ephemeral feelings, varying in intensity and duration that usually involves more than one emotion” (See, for example, Lane, A. M. and Terry, P. C., 2000. The nature of mood: Development of a conceptual model with a focus on depression. Journal of Applied Sport Psychology, 12(1), pp. 16-33). Various measures of mood have been established, including the Brunel Mood Scale (“BRUMS” scale) (See, for example, Terry, P. C., Lane, A. M., & Fogarty, G. J. (2003). Construct validity of the POMS-A for use with adults. Psychology of Sport and Exercise, 4, 125-139; and Terry, P. C., Lane, A. M., Lane, H. J., & Keohane, L. (1999). Development and validation of a mood measure for adolescents. Journal of Sports Sciences, 17, 861-872), and the Profile of Mood States (“POMS” scale) (See, for example, McNair, D. M., Lorr, M., & Droppelman, L. F. (1971). Manual for the Profile of Mood States. San Diego: Educational and Industrial Testing Services). The BRUMS scale includes a 24-item questionnaire of mood descriptors, that inform six subscales: anger, confusion, depression, fatigue, tension, and vigour. A mood-altering cannabinoid is a cannabinoid that causes mood alterations when administered to a subject.

As used herein, “impairment” means any loss or abnormality of psychological, physiologic, or anatomic structure or function. An “impairing cannabinoid” is a cannabinoid that causes impairment when administered to a subject. As used herein, “cognitive impairment” means dysfunction in a number of levels in a subject from motor coordination to complex tasks, such as the ability to plan, organize, solve problems, make decisions, remember and control emotions and behavior (See, for example, Crean, R. D., Crane, N. A. and Mason, B. J., 2011. An evidence based review of acute and long-term effects of cannabis use on executive cognitive functions. Journal of Addiction Medicine, 5(1), p.1).

As used herein, the term “subject” refers to any lifeform comprising an endocannabinoid system, including vertebrates and chordate invertebrates. In some embodiments, the subject may be a vertebrate or chordate invertebrate. In some embodiments, the subject may be a vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human, a mouse, a cat, a dog, or a cat. In some embodiments, the subject is a human.

As used herein, the term “vaporization” refers to a process by which a material undergoes at least one phase transition to enter into a gaseous phase, as a gas, or as liquid droplets or solid particulates suspended in a gas. Unless context dictates otherwise, vaporization includes evaporation, boiling and aerosolization.

As used herein, the term “vapor” refers to a gas or a gaseous mixture including liquid droplets and/or solid particulates suspended in the gas.

### Combinations of CBC-Type and THC-Type Cannabinoids

It was surprisingly found that CBC-type cannabinoids can modulate the effects of THC-type cannabinoids, particularly the high, and more particularly, the mood-altering and impairment effects of THC-type cannabinoids, when administered to or consumed by a subject.

According to an aspect of the disclosure, there is provided combinations of a CBC-type cannabinoid and a THC-type cannabinoid. As will be discussed in more detail herein, when administered to or consumed by a subject, the THC-type cannabinoid induces a high and the CBC-type cannabinoid modulates the high.

Delta-9-THC is the primary psychoactive compound found in cannabis. Delta-9-THC acts on the endocannabinoid system, and is a partial agonist of the CB₁ and CB₂ cannabinoid receptors. When administered to a subject, delta-9-THC may produce physiological and psychological effects, including the “high” typically ascribed to delta-9-THC. The “high” associated with delta-9-THC is a combination of psychological and physiological factors that result in an altered state of mind. These effects include mood alteration, impairment, or both. Certain cannabinoids are known to produce euphoria and relaxation, with increased dosages expressing as depressant effects. Desirable subjective effects of the “high” can include feelings of tranquility and auditory and visual sensory alterations. Other effects of the “high” can include impairment in prospective memory and executive function, expressing as impaired short-term memory and attention, performance of complex tasks, judgment, motor skills and slowed reaction times. The “high” can also result in perceptual alterations such as heightened sense perception and altered sense of time. Additionally, the “high” can alter the social interactions an afflicted user may have, for example, modulating the paranoia and anxiety that a user experiences. See, for example, Wrege, J., Schmidt, A., Walter, A., Smieskova, R., Bendfeldt, K., Radue, E.W., E Lang, U. and Borgwardt, S., 2014. Effects of cannabis on impulsivity: a systematic review of neuroimaging findings. Current Pharmaceutical Design, 20(13), pp. 2126-2137.; Ameri, A., 1999. The effects of cannabinoids on the brain. Progress in Neurobiology, 58(4), pp. 315-348; Green, B. O. B., Kavanagh, D. and Young, R., 2003. Being stoned: a review of self-reported cannabis effects. Drug and Alcohol Review, 22(4), pp. 453-460; and Foltin, R. W., Brady, J. V., Fischman, M. W., Emurian, C. S. and Dominitz, J., 1987. Effects of smoked marijuana on social interaction in small groups. Drug and Alcohol Dependence, 20(1), pp. 87-93).

Generally, physiological effects are mainly related to organs and systems function, such as those related to enzymatic driven metabolic functions, respiration, digestion, muscle and bone structure coordination connected to locomotion, blood circulation, etc. A common method for evaluating the physiological effects of cannabinoids is the tetrad test (see for example, Metna-Laurent, M., Mondésir, M., Grel, A., Vallée, M. and Piazza, P. V., 2017. Cannabinoid-induced tetrad in mice. Current Protocols in Neuroscience, 80(1), pp. 9-59). In the tetrad test, rodents are administered compounds and the effect on hypolocomotion, catalepsy, hypothermia and antinociception is evaluated. If these effects are observed following treatment and are not observed when also treated with antagonists or inverse agonists, such as rimonabant, the compound is considered to exhibit cannabinoid receptor-mediated effects. If effects are not observed in any one factor following treatment, or the effects are all observed but are not affected by treatment with antagonists or inverse agonists, the compound is not considered to exhibit cannabinoid receptor-mediated effects.

Psychological effects can include feelings of euphoria, alterations to emotions, moods, motivation, personality, interpersonal relationships, perception, etc. Although there may be overlaps between measurable physiological effects and psychological effects, evaluation of psychological effects may require subjective measures. As such, relying solely on purely objective physical measurements, such as the tetrad test, without the self-assessment from the subject may be insufficient to evaluate certain psychological effects, and indeed, the totality of the effects of a cannabinoid (See, for example, Monory, K., Blaudzun, H., Massa, F., Kaiser, N., Lemberger, T., SchQtz, G., Wotjak, C. T., Lutz, B. and Marsicano, G., 2007. Genetic dissection of behavioural and autonomic effects of Δ9-tetrahydrocannabinol in mice. PLoS Biology, 5(10), p.e269). For example, it may not be possible to evaluate a rodent's “anger” based on objective measurements.

Other THC-type cannabinoids may also induce the “high” associated with delta-9-THC. Delta-8-THC has been shown to also produce effects that are similar to delta-9-THC (See, for example, Adams, R., 1942. Marihuana: Harvey lecture, Feb. 19, 1942. Bulletin of the New York Academy of Medicine, 18(11), p.705.; and Hollister, L. E. and Gillespie, H. K., 1973. Delta-8- and delta-9-tetrahydrocannabinol; Comparison in man by oral and intravenous administration. Clinical Pharmacology & Therapeutics, 14(3), pp. 353-357). Similarly, delta-9-THCP has also been shown to produce effects that are similar to delta-9-THC (Citti, C., Linciano, P., Russo, F., Luongo, L., lannotta, M., Maione, S., Laganà, A., Capriotti, A. L., Forni, F., Vandelli, M. A. and Gigli, G., 2019. A novel phytocannabinoid isolated from Cannabis sativa L. with an in vivo cannabimimetic activity higher than Δ9-tetrahydrocannabinol: Δ9-Tetrahydrocannabiphorol. Scientific Reports, 9(1), pp. 1-13). Pre-clinical studies show that hexahydrocannabinol (HHC) produces cannabinoid effects in animals (Edery, H., Grunfeld, Y., Ben-Zvi, Z. and Mechoulam, R., 1971. Structural requirements for cannabinoid activity. Annals of the New York Academy of sciences, 191(1), pp. 40-53). THC-OAc was found to produce a 2-fold greater degree of ataxia (lack of coordination and balance as a result of sensory dysfunction) in dogs compared with Δ9-THC (See, for example, National Research Council, 1985. Possible Long-Term Health Effects of Short-Term Exposure to Chemical Agents, Volume 3: Final Report: Current Health Status of Test Subjects. National Academies Press). Without being bound by theory, it is believed that THC-type cannabinoids, which exhibit similar structures to delta-9 THC, will exhibit receptor binding characteristics similar to delta-9-THC (e.g., being agonists of CB1 and CB2 receptors (“CB1R” and “CB2R”, respectively)), will similarly induce a “high”.

In some embodiments, the THC-type cannabinoid includes tetrahydrocannabinol (THC), its analogs with tails of different alkyl chain lengths including tetrahydrocannabivarinol (THCV), tetrahydrocannabibutol (THCB), tetrahydrocannabihexol (THCH), tetrahydrocannabiphorol (THCP), their hydrogenated analogs including hexahydrocannabinol (HHC), hexahydrocannabivarinol (HHCV), hexahydrocannabibutol (HHCB), hexahydrocannabihexol (HHCH), hexahydrocannabiphorol (HHCP), their oxidized degradants including cannabinol (CBN), cannabivarinol (CBNV), cannabibutol (CBNB), cannabihexol (CBNH), cannabiphorol (CBNP), their acyl esters including tetrahydrocannabinol-o-acetate (THC-O-Ac), tetrahydrocannabivarinol-o-acetate (THCV-O-Ac), tetrahydrocannabibutol-o-acetate (THCB-O-Ac), tetrahydrocannabihexol-o-acetate (THCH-O-Ac), tetrahydrocannabiphorol-o-acetate (THCP-O-Ac), their hydrogenated derivatives 11-hydroxy-tetrahydrocannabinol (11-OH-THC), 11-hydroxy-tetrahydrocannabivarinol (11-OH-THCV), 11-hydroxy-tetrahydrocannabibutol (11-OH-THCB), 11-hydroxy-tetrahydrocannabihexol (11-OH-THCH), 11-hydroxy-tetrahydrocannabiphorol (11-OH-THCP) or a combination thereof. In some embodiments, the THC-type cannabinoid includes THC. In some embodiments, the THC includes delta-8 THC, delta-9 THC, or both. In some embodiments, the THC includes delta-9 THC. Without being bound by theory, it is believed that the acidic forms of THC-type cannabinoids have low activity on cannabinoid receptors, and have limited ability to induce the “high” associated with cannabis. However, the acidic forms of THC-type cannabinoids can be decarboxylated prior to or even during use, such as by thermal degradative processes, to provide the “high”.

Non-THC-type compounds may modulate the effects of THC-type compounds such as delta-9-THC. For example, compounds that are cannabinoid receptor ligands may affect the binding of THC-type cannabinoids to cannabinoid receptors. The inverse agonist/antagonist of CB1R rimonabant has been shown to prevent the tetrad effects of delta-9-THC (See, for example, Metna-Laurent, supra) by competitively binding to CB1R. Cannabidiol has also been shown to affect the effects of THC (See, for example Boggs, D. L., Nguyen, J. D., Morgenson, D., Taffe, M. A. and Ranganathan, M., 2018. Clinical and preclinical evidence for functional interactions of cannabidiol and Δ9-tetrahydrocannabinol. Neuropsychopharmacology, 43(1), pp. 142-154). Other ways non-THC-type compounds include affecting metabolism of THC-type compounds (e.g., by inhibiting cytochrome P450 enzyme activity on THC-type compounds), affecting bioavailability of THC-type compounds (e.g., by acetylation or emulsification in a SMED/SNED system), and other methods known to those skilled in the art.

CBC is a cannabinoid that exhibits partial CB1R and CB2R agonist activity (See, for example, Zagzoog, A., Mohamed, K. A., Kim, H. J. J., Kim, E. D., Frank, C. S., Black, T., Jadhav, P. D., Holbrook, L. A. and Laprairie, R. B., 2020. In vitro and in vivo pharmacological activity of minor cannabinoids isolated from Cannabis sativa. Scientific Reports, 10(1), pp. 1-13) and has also been shown to modulate certain effects of THC (DeLong, G. T., Wolf, C. E., Poklis, A. and Lichtman, A. H., 2010. Pharmacological evaluation of the natural constituent of Cannabis sativa, cannabichromene and its modulation by Δ9-tetrahydrocannabinol. Drug and Alcohol Dependence, 112(1-2), pp. 126-133 (DeLong).

However, attempts to document the effects of CBC on a user's “high” have been conflicting. DeLong discloses experiments conducted on rodents to explore potential tetrad effects of CBC, and how THC modulates the effects of CBC. DeLong discloses that intravenously administered CBC produced locomotor suppression, catalepsy, and hypothermia consistently at high doses (30 and 100 mg/kg), but only occasionally produced a small magnitude of antinociception. The tetrad effects were not blocked by rimonabant. When co-administered with an inactive dose of THC (0.3 mg/kg), tetrad effects other than locomotor suppression were modulated. Further, the co-administration of THC and CBC led to increased THC brain levels, but not THC blood levels or CBC brain or blood levels. DeLong speculated that “it is unlikely that CBC by itself relevantly contributes to the psychoactive effects of marijuana”. Further, DeLong speculated that “high doses of CBC may augment a threshold dose of THC to produce significant cannabinoid activity in the tetrad”. DeLong fails to disclose how the effects of increased of THC brain levels might interact with competitive CB1R and CB2R binding in an active dose of THC, how different routes of administration may affect synergistic effects, what happens to the synergistic effect when active doses of THC are used, or how the interaction of THC and CBC affects the high. In contrast, Ilan et al. examined if systematic variations in the concentrations of THC (1.91%-3.09%), CBD and CBC (0.12%-0.6%) in smoked marijuana modulated the subjective, behavioral or neurophysiological effects in healthy volunteers and found that varying the concentration of CBC did not change significantly any of the subjective, physiological or performance indicators (See, for example, Ilan, A. B., Gevins, A., Coleman, M., ElSohly, M. A. and De Wit, H., 2005. Neurophysiological and subjective profile of marijuana with varying concentrations of cannabinoids. Behavioural Pharmacology, 16(5-6), pp. 487-496).

In a recent study, Moore et al. investigated potential interoceptive changes when minor cannabinoids, including cannabichromene (CBC), were administered with a set dose of delta-9-tetrahydrocannabinol (D9-THC) to Long Evans rats (Moore, C. F., Marusich, J., Haghdoost, M., Lefever, T. W., Bonn-Miller, M. O. and Weerts, E. M., 2023. Evaluation of the Modulatory Effects of Minor Cannabinoids and Terpenes on Delta-9-Tetrahydrocannabinol Discrimination in Rats. Cannabis and Cannabinoid Research, 8(S1), pp. S42-S50). Following intraperitoneal injection of D9-THC at 3 mg/kg, CBC was administered at 3-30 mg/kg; trained rats were then evaluated for response rates and perceptual effects. The tested doses were found to decrease response rates but did notsignificantly alter the perceptual effects of D9-THC. The authors acknowledged limitations in their methodology: dosage range, influenced by scant existing literature, may not have been optimal for modulation of the effects of D9-THC; and use of a uniform D9-THC dose across all experimental conditions may have hidden insights gained by the use of a range of doses. The study also relied on drug discrimination methods to gauge the effects, a complex approach that may not fully encapsulate the intricate interactions between cannabinoids.

Two studies have assessed the pharmacokinetics (PK) of CBC. In 2021, Peters et al. examined the plasma levels of CBC, cannabidiol (CBD), and D9-THC in humans after administering an oil blend (CBD:CBC:D9-THC ratio of 20:1.1:0.9) for seven days (Peters, E. N., MacNair, L., Mosesova, I., Christians, U., Sempio, C., Klawitter, J., Land, M. H., Ware, M. A., Turcotte, C. and Bonn-Miller, M. O., 2022. Pharmacokinetics of cannabichromene in a medical cannabis product also containing cannabidiol and Δ9-tetrahydrocannabinol: a pilot study. European Journal of Clinical Pharmacology, pp. 1-7). Despite the CBD dosage being ˜18 times higher than that of CBC and D9-THC, the plasma concentration of CBD—as measured by the Area Under the Curve (AUC) between time 0 and the last detectable value—was only 6.6 to 9.8 times greater relative to the initial dose concentration than that of CBC. CBC also showed higher plasma prevalence compared to D9-THC. These findings indicate possible preferential absorption of CBC compared to CBD and THC when combined in an oil blend. In 2023, a separate study by Moore et al. focused on the pharmacokinetics of several minor cannabinoids, including CBC, discretely administered to Sprague-Dawley rats through oral gavage once daily for 14 days (Moore, C. F., Weerts, E. M., Kulpa, J., Schwotzer, D., Dye, W., Jantzi, J., McDonald, J. D., Lefever, T. W. and Bonn-Miller, M. O., 2023. Pharmacokinetics of oral minor cannabinoids in blood and brain. Cannabis and cannabinoid research, 8(S1), pp. S51-S61). Of the four cannabinoids tested, CBC exhibited the highest maximum observed concentration and AUC, indicating substantial presence in plasma during and post-treatment. Interestingly, CBC showed the highest concentrations in brain tissue, suggesting its effective penetration of the blood-brain barrier and persistence in the brain.

Without being bound by theory, it is believed that when administered to or consumed by a subject, CBC modulates the effects of the THC-type cannabinoid by binding to alternative sites on the endocannabinoid system receptors, while concurrently acting on other systems. For example, CBC may potentiate adenosine signaling and have an effect on anandamide levels, which may contribute to effects on mood like elevating moods. In addition, CBC exhibits TRPA1 channel agonism, which may contribute to analgesic and anti-tensive effects, and relaxation.

Without being bound by theory, it is believed that CBC-type cannabinoids, which have structures similar to CBC, will similarly modulate the effects of the THC-type cannabinoid when administered to or consumed by a subject. In some embodiments, the CBC-type cannabinoid is cannabichromevarin (CBCV), cannabichromebutol (CBCB), cannabichromene (CBC), cannabichromehexol (CBCH), cannabichromephorol (CBCP), or a combination thereof. In some embodiments, the CBC-type cannabinoid is CBC.

In some embodiments, the CBC-type cannabinoid modulates the high induced by the THC-type cannabinoid. In some embodiments, the CBC-type cannabinoid modulates the mood-alteration, impairment, or both, of the THC-cannabinoid.

In some embodiments, the CBC-type cannabinoid modulates the onset time of impairment, the onset time of mood alteration, sensory perception, executive function, sociability, anger, confusion, depression, fatigue, despair, tension, vigour, grit, resiliency, sociability, willingness to explore (e.g., adventure seeking) or a combination thereof.

In some embodiments, the modulation of impairment of a subject comprises modulation of onset time of impairment; sensory perception; executive function (e.g. cognition); memory; or any combination thereof. In some embodiments, the CBC-type cannabinoid modulates the impairment induced by the THC-type cannabinoid by decreasing onset time of impairment, increases the sensory perception of the subject, increases the executive function (e.g. cognition) of the subject, improves the memory of the subject, or a combination thereof.

The impairment of a subject may be assessed by tests known to skilled persons in the art, including, for example, a field sobriety test (e.g., horizontal gaze nystagmus test, walk-and-turn test and/or one-leg stand test), pupil dilation, Cognitive Drug Research (CDR) System, and Rey-Osterrieth complex figure.

In some embodiments, the CBC-type cannabinoid modulates the duration of impairment induced by the THC-type cannabinoid by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100%. In some embodiments, the CBC-type cannabinoid increases the duration of impairment induced by the THC-type cannabinoid by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100%. Methods for detecting the duration of impairment induced by the THC-type cannabinoid include periodic biofluid collection to monitor blood THC concentrations, periodic subjective measures (e.g., using a Likert scale), periodic cognitive and psychomotor tests, eye tracking or a combination thereof (See for example, Grabenauer, M and Wilson J, 2020. Differences in Cannabis Impairment and its Measurement Due to Route of Administration; and Wurz, G. T., DeGregorio, M. W. Indeterminacy of cannabis impairment and Δ⁹-tetrahydrocannabinol (Δ⁹-THC) levels in blood and breath. Sci Rep 12, 8323 (2022)). An analysis of 80 scientific studies has identified a ‘window of impairment’ of between three and 10 hours caused by moderate to high doses of THC. The study found the exact duration of impairment depends on the dose of THC, whether the THC is inhaled or taken orally, whether the cannabis user is regular or occasional and the demands of the task being undertaken while intoxicated (McCartney, D., Arkell, T. R., Irwin, C. and McGregor, I. S., 2021. Determining the magnitude and duration of acute Δ9-tetrahydrocannabinol (Δ9-THC)-induced driving and cognitive impairment: a systematic and meta-analytic review. Neuroscience & Biobehavioral Reviews, 126, pp. 175-193).

In some embodiments, the CBC-type cannabinoid modulates the sensory perception (e.g., sound, smell, sight, taste, touch, proprioception, balance) of a subject that has been administered or has consumed the THC-type cannabinoid. Visual function assessments can be performed, for example, by using optical devices to measuring visual parameters such as statis visual acuity, contrast sensitivity, accommodative response, etc. (See, Ortiz-Peregrina, S., Ortiz, C., Casares-López, M., Jiménez, J. R. and Anera, R. G., 2021. Effects of cannabis on visual function and self-perceived visual quality. Scientific Reports, 11(1), pp. 1-11). Taste intensity perception can be assessed, for example, by measuring appetite, and subjective effects using visual analog scale (de Bruijn, S.E., de Graaf, C., Witkamp, R. F. and Jager, G., 2017, Explorative Placebo-Controlled Double-Blind Intervention Study with Low Doses of Inhaled Δ9-Tetrahydrocannabinol and Cannabidiol Reveals No Effect on Sweet Taste Intensity Perception and Liking in Humans, Cannabis and Cannabinoid Research, 2.1, pp. 114-122). Other sensory measurements are known to those skilled in the art (See, for example, Zuardi, W., Atakan, Z. and McGuire, P. K., 2011, Modulation of auditory and visual processing by Delta-9-Tetrahydrocannabinol and Cannabidiol: an fMRI study. Neuropsychopharmacology, 36(7), pp. 1340-1348; National Center for Health Statistics, Centers for Disease Control and Prevention, National Health and Nutrition Examination Survey (NHANES): Taste and Smell Examination Component Manual (2013); Douglas, J. E., Mansfield, C. J., Arayata, C. J., Cowart, B. J., Colquitt, L. R., Maina, I. W., Blasetti, M. T., Cohen, N. A. and Reed, D. R., 2018. Taste exam: a brief and validated test. JoVE (Journal of Visualized Experiments), (138), p.e56705; Kostek, M., Polaski, A., Kolber, B., Ramsey, A., Kranjec, A. and Szucs, K., 2016. A protocol of manual tests to measure sensation and pain in humans. JoVE (Journal of Visualized Experiments), (118), p.e54130).

In some embodiments, the CBC-type cannabinoid modulates the executive function of a subject that has been administered or has consumed a THC-type cannabinoid. In some embodiments, the CBC-type cannabinoid increases the executive function of a subject that has been administered or has consumed a THC-type cannabinoid as compared to a subject that has not been administered or has not consumed a CBC-type cannabinoid. A relative increase in the executive function during a “high” may be desired by certain subjects who wish to experience euphoria, relaxation, analgesia, or other effects while being less impacted in their executive functions. A relative decrease the executive function during a “high” may be desired by other subjects. Examples of tests to determine executive functioning include cognitive and psychomotor performance assessments. Tests that have been used to determine how cannabis affects executive function include The Stroop Color Word Test, the Trail Making Test, the Wisconsin Card Sorting Test (WCST), and Letter-Number Sequencing subtest of the Wechsler Adult Intelligence Scale (WAIS) (See, Gruber, S. A., Sagar, K. A., Dahlgren, M. K., Racine, M. T., Smith, R. T. and Lukas, S. E., 2016. Splendor in the grass? A pilot study assessing the impact of medical marijuana on executive function. Frontiers in Pharmacology, p.355). Other tests for evaluating executive function are known to those skilled in the art (See, for example, see Crean, R. D., Crane, N. A. and Mason, B. J., 2011. An evidence-based review of acute and long-term effects of cannabis use on executive cognitive functions. Journal of Addiction Medicine, 5(1), p.1; and Faria, C. D. A., Alves, H. V. D. and Charchat-Fichman, H., 2015. The most frequently used tests for assessing executive functions in aging. Dementia & Neuropsychologia, 9, pp. 149-155).

In some embodiments, the modulation of the mood alteration in a subject comprises modulation of: duration of the mood alteration; sociability; anger; confusion; depression; fatigue; despair; tension; vigour; grit; resiliency; sociability; or any combination thereof.

The mood of a subject may be assessed by POMS, BRUMS, or other measures known to skilled persons in the art. A baseline or a “negative control” may first be established in a subject or a group of subjects without cannabinoid use. Following administration of one or more cannabinoids, the mood of a subject may be assessed at predetermined intervals to determine when the subject's mood deviates from the baseline. In determining how CBC-type cannabinoids, such as CBC, modulates the high associated with THC-type cannabinoids, such as delta-9-THC, a “positive control” where only delta-9-THC is administered may be created.

In drug testing with rodents, several behavioral tests are pivotal for evaluating mood-related outcomes, particularly involving anxiety and depression. The Open Field Test gauges anxiety-like behavior and locomotor activity by observing a mouse's preference for staying close to walls versus exploring a large, open box. The Y-Maze, typically used for assessing spatial memory, can also infer anxiety levels based on the mouse's choice between enclosed and open arms. The Forced Swim Test is a standard method to evaluate depressive-like behavior, where a mouse's time spent immobile in water is indicative of ‘behavioral despair’, akin to depression. Lastly, the Elevated Plus Maze measures anxiety by recording the mouse's willingness to venture onto exposed arms versus staying in enclosed areas. Collectively, these tests can provide insights into the psychological impacts of drugs, particularly in the realms of anxiety and depression in rodents. Without wishing to be bound by any theory, it is believed that behavioral outcomes in such tests can be used as a functional model or proxy for human mood. For example, when utilizing a Y-maze test, an increased willingness to explore may be used as a proxy for elevated mood because it suggests at a functional or neurological level that (i) the animal is able to evaluate a particular goal and make a decision; (ii) the animal is able to take action where ethe emotion gives priority or urgency; and (iii) the animal displays physiological changes, and then behavioral action.

In some embodiments, the CBC-type cannabinoid increases the duration of mood-alteration induced by the THC-type cannabinoid in a subject by at least 1%, 2%, 3%, 4%, 5%, 6%, 7%, 8%, 9%, 10%, 15%, 20%, 25%, 30%, 35%, 40%, 45%, 50%, 60%, 70%, 80%, 90%, or 100%.

In some embodiments, the CBC-type cannabinoid induces a decrease in a subject's anger score on the BRUMS scale as compared to a THC-type cannabinoid control. A decrease in a subject's anger score indicates increased sociability, friendliness and connection to others, while an increase in a subject's score would indicate increased irritability and hostility towards others. A subject who wants to use cannabis in a social setting or alleviate social anxiety may prefer a high where the anger score is decreased, resulting in a modulated high with increased sociability, friendliness and connection to others. In contrast, a subject desirous of a solo, introspective experience may prefer a high where the anger score is unchanged or even increased.

In some embodiments, the CBC-type cannabinoid induces a modulated confusion score on the BRUMS scale as compared to a THC-type cannabinoid control. In some embodiments, the CBC-type cannabinoid induces a decreased confusion score on the BRUMS scale as compared to a THC-type cannabinoid control. A subject who wants to experience a clear-headed, focused high may prefer a high where the confusion score is decreased, resulting in a modulated high with increased sociability, friendliness and connection to others. In contrast, a subject desirous of a more intoxicating experience may prefer a high where the confusion score is increased. Additionally, the confusion score tends to show a negative correlation with the executive function of the subject such that a decrease in executive function may indicate an increase of confusion and vice versa.

In some embodiments, the CBC-type cannabinoid induces a modulation in depression score on the BRUMS scale as compared to a THC-type cannabinoid control. In some embodiments, the CBC-type cannabinoid induces a decreased depression score on the BRUMS scale as compared to a THC-type cannabinoid control. A subject wanting to experience a happier high may prefer a high where the depression score is decreased. Generally, a subject does not prefer an increase in depression score, as it would indicate increased sadness/unhappiness.

In some embodiments, the CBC-type cannabinoid induces a modulation in fatigue score on the BRUMS scale as compared to a THC-type cannabinoid control. In some embodiments, the CBC-type cannabinoid induces a decrease in fatigue score on the BRUMS scale as compared to a THC-type cannabinoid control. A subject wanting to experience an invigorating, “daytime” high may prefer a high where the fatigue score is decreased. In contrast, a subject wanting to experience a soporific or restful high may prefer a high where the fatigue score is increased.

In some embodiments, the CBC-type cannabinoid induces a modulation in tension score on the BRUMS scale as compared to a THC-type cannabinoid control. Certain subjects may exhibit increased productivity and/or focus when under some tension. A subject wanting to experience a productive or focused high may prefer an increased tension score. However, too much tension may induce feelings of anxiety (such as generalized anxiety or panic anxiety) and worry. A subject wanting to experience a more relaxed, calm high or with anxiolytic properties may prefer a high where the tension score is decreased.

In some embodiments, the CBC-type cannabinoid induces a modulation in vigor score on the BRUMS scale as compared to a THC-type cannabinoid control. In some embodiments, the CBC-type cannabinoid induces an increase in vigor score on the BRUMS scale as compared to a THC-type cannabinoid control. A subject wanting to experience a more uplifting, energetic high may prefer a high with increased vigor scores. In contrast, a subject wanting to experience a more relaxing, soporific high may prefer a high with decreased vigor scores.

In some embodiments, the CBC-type cannabinoid induces a modulation of sociability in a subject as compared to a THC-type cannabinoid control. In some embodiments, the CBC-type cannabinoid induces an increased sociability as compared to a THC-type cannabinoid control. Sociability may be measured by using the BRUMS anger score or another sociability assessment. Increased sociability indicates increased friendliness and enjoyment when interacting with others. This may be desirable for in social settings, especially if a subject is afflicted with social anxiety. In contrast, a subject wanting a more introspective high may prefer decreased sociability.

In some embodiments, the CBC-type cannabinoid is adapted to be included with the THC-type cannabinoid (either in a single dosage form or a single product (e.g., single consumable product) or in separate dosage forms or separate products (e.g., separate consumable products)) in a ratio of CBC-type cannabinoid to THC-type cannabinoid of from 1:1 to 100:1. In still other aspects, the composition further comprises a CBC-type cannabinoid:THC-type cannabinoid ratio (CBC:THC). of from 1:2 to 100:1. In still other aspects, the composition further comprises a CBC-type cannabinoid:THC-type cannabinoid ratio (CBC:THC) of from 1:1 to 100:1 with the proviso that when the cannabinoid:THC-type cannabinoid (CBC:THC) ratio is 1.1:0.9 and cannabidiol (CBD) is present in the composition, the ratio of CBD:CBC:THC is not 20:1.1:0.9 (CBD:CBC:THC). In still yet further aspects, the CBC-type cannabinoid:THC-type cannabinoid ratio (CBC:THC) is from 1:3 to 20:1. In still yet further aspects, the CBC-type cannabinoid:THC-type cannabinoid ratio (CBC:THC) is from 1:4 to 15:1. In still yet further aspects, the CBC-type cannabinoid:THC-type cannabinoid ratio is from 1:5 to 10:1. In still yet further aspects, the CBC-type cannabinoid:THC-type cannabinoid ratio is from 1:6 to 5:1. In still yet further aspects, the CBC-type cannabinoid:THC-type cannabinoid ratio (CBC:THC) is from 1:7 to 4:1.

In yet still other aspects, the CBC-type cannabinoid: THC-type cannabinoid ratio (CBC:THC) is about 20:1. In yet still other aspects, the CBC-type cannabinoid: THC-type cannabinoid ratio (CBC:THC) is about 19:1. In yet still other aspects, the CBC-type cannabinoid: THC-type cannabinoid ratio (CBC:THC) is about 18:1. In yet still other aspects, the CBC-type cannabinoid: THC-type cannabinoid ratio (CBC:THC) is about 17:1. In yet still other aspects, the CBC-type cannabinoid: THC-type cannabinoid ratio (CBC:THC) is about 16:1. In yet still other aspects, the CBC-type cannabinoid: THC-type cannabinoid ratio (CBC:THC) is about 15:1. In yet still other aspects, the CBC-type cannabinoid: THC-type cannabinoid ratio (CBC:THC) is about 14:1. In yet still other aspects, the CBC-type cannabinoid: THC-type cannabinoid ratio (CBC:THC) is about 13:1. In yet still other aspects, the CBC-type cannabinoid: THC-type cannabinoid ratio (CBC:THC) is about 12:1. In yet still other aspects, the CBC-type cannabinoid: THC-type cannabinoid ratio (CBC:THC) is about 11:1. In yet still other aspects, the CBC-type cannabinoid: THC-type cannabinoid ratio (CBC:THC) is about 10:1. In yet still other aspects, the CBC-type cannabinoid: THC-type cannabinoid ratio (CBC:THC) is about 9:1. In yet still other aspects, the CBC-type cannabinoid: THC-type cannabinoid ratio (CBC:THC) is about 8:1. In yet still other aspects, the CBC-type cannabinoid: THC-type cannabinoid ratio (CBC:THC) is about 7:1. In yet still other aspects, the CBC-type cannabinoid: THC-type cannabinoid ratio (CBC:THC) is about 6:1. In yet still other aspects, the CBC-type cannabinoid: THC-type cannabinoid ratio (CBC:THC) is about 5:1. In yet still other aspects, the CBC-type cannabinoid: THC-type cannabinoid ratio (CBC:THC) is about 4:1. In yet still other aspects, the CBC-type cannabinoid: THC-type cannabinoid ratio (CBC:THC) is about 3:1. In yet still other aspects, the CBC-type cannabinoid: THC-type cannabinoid ratio (CBC:THC) is about 2:1. In still yet other aspects, the CBC-type cannabinoid:THC-type cannabinoid ratio (CBC:THC) is about 1:2. In still yet other aspects, the CBC-type cannabinoid:THC-type cannabinoid ratio (CBC:THC) is about 1:3. In still yet other aspects, the CBC-type cannabinoid:THC-type cannabinoid ratio (CBC:THC) is about 1:4. In still yet other aspects, the CBC-type cannabinoid:THC-type cannabinoid ratio (CBC:THC) is about 1:5. In still yet other aspects, the CBC-type cannabinoid:THC-type cannabinoid ratio (CBC:THC) is about 1:6. In still yet other aspects, the CBC-type cannabinoid:THC-type cannabinoid ratio (CBC:THC) is about 1:7 In still yet other aspects, the CBC-type cannabinoid:THC-type cannabinoid ratio (CBC:THC) is about 1:8. In still yet other aspects, the CBC-type cannabinoid:THC-type cannabinoid ratio (CBC:THC) is about 1:9. In still yet other aspects, the CBC-type cannabinoid:THC-type cannabinoid ratio (CBC:THC) is about 1:10. In still yet other aspects, the CBC-type cannabinoid:THC-type cannabinoid ratio (CBC:THC) is about 1:11. In still yet other aspects, the CBC-type cannabinoid:THC-type cannabinoid ratio (CBC:THC) is about 1:12. In still yet other aspects, the CBC-type cannabinoid:THC-type cannabinoid ratio (CBC:THC) is about 1:13. In still yet other aspects, the CBC-type cannabinoid:THC-type cannabinoid ratio (CBC:THC) is about 1:14. In still yet other aspects, the CBC-type cannabinoid:THC-type cannabinoid ratio (CBC:THC) is about 1:15. In still yet other aspects, the CBC-type cannabinoid:THC-type cannabinoid ratio (CBC:THC) is about 1:16. In still yet other aspects, the CBC-type cannabinoid:THC-type cannabinoid ratio (CBC:THC) is about 1:17. In still yet other aspects, the CBC-type cannabinoid:THC-type cannabinoid ratio (CBC:THC) is about 1:18. In still yet other aspects, the CBC-type cannabinoid:THC-type cannabinoid ratio (CBC:THC) is about 1:19. In still yet other aspects, the CBC-type cannabinoid:THC-type cannabinoid ratio (CBC:THC) is about 1:20.

In some embodiments, the CBC-type cannabinoid can be: cannabigerol (CBG), cannabicyclol (CBL), cannabicitran (CBT), isoprenyl-CBC, sesqui-CBC, sesqui-CBG, or any combinations thereof.

In some embodiments, the CBC-type cannabinoid, the THC-type cannabinoid or both the CBC-type cannabinoid and THC-type cannabinoid can be formulated for oral administration, buccal administration, intravenous administration, peritoneal administration, intradermal administration, pulmonary and/or inhalation administration, topical administration, transmucosal administration, rectal administration, or any combination thereof. In some embodiments, the CBC-type cannabinoid, the THC-type cannabinoid or both the CBC-type cannabinoid and THC-type cannabinoid is formulated for oral or buccal administration. In some embodiments, the CBC-type cannabinoid, the THC-type cannabinoid or both the CBC-type cannabinoid and THC-type cannabinoid is formulated for pulmonary administration. In some embodiments, the CBC-type cannabinoid, the THC-type cannabinoid or both the CBC-type cannabinoid and THC-type cannabinoid formulated for oral or buccal administration.

### Compositions Containing CBC-Type Cannabinoids

According to another aspect, the present disclosure provides compositions comprising a CBC-type cannabinoid, a THC-type cannabinoid, or both a CBC-type cannabinoid and a THC-cannabinoid. In some embodiments the CBC-type cannabinoid is provided or formulated in a single composition without any THC-type cannabinoids. In some aspects, the THC-type cannabinoid is provided or formulated without any CBC-type cannabinoids. In yet further aspects, the CBC-type cannabinoids and the THC-type cannabinoids are formulated together, in combination, as a single composition. In some aspects, the composition is one or more dosage forms. In other aspects, the composition is one or more products (e.g., such as a consumable product).

When the CBC-type cannabinoid and the THC-type cannabinoid are provided in separate compositions, they can be administered to or consumed by a subject in any order, either simultaneously, concurrently, or sequentially. In other words, the order in which the compositions are administered to or consumed by a subject is not critical.

In some embodiments, the one or more compositions (e.g., which can be a first single (e.g., one) composition containing the CBC-type cannabinoid and a second, single (e.g., one) composition containing the THC-type cannabinoid, or a composition containing both the CBC-type cannabinoid and the THC-type cannabinoid (e.g., as a combination)), when administered to or consumed by a subject, provide a modulated high as compared to a control composition that does not contain the CBC-type cannabinoid (or is not administered to a subject if the CBC-type cannabinoid and THC-type cannabinoid are contained in separate (e.g., individual) compositions and administered separately).

In some embodiments, the modulated high in a subject includes a modulated impairment, mood alteration, or both, as compared to the control composition. In some embodiments, the modulated impairment includes a modulated: impairment onset time; sensory perception; executive function; or any combination thereof as compared to a control composition without the CBC-type cannabinoid.

In some embodiments, the modulated mood alteration in a subject includes a modulated: mood alteration onset time; sociability; anger; confusion; depression; fatigue; despair; tension; vigor; grit; resiliency; sociability; willingness to explore (e.g., adventure seeking) or any combination thereof as compared to a control composition without the CBC-type cannabinoid.

In some embodiments, the composition has a CBC-type cannabinoid:THC-type cannabinoid ratio of from 1:1 to 100:1. In still other aspects, the composition further comprises a CBC-type cannabinoid:THC-type cannabinoid ratio (CBC:THC) of from 1:1 to 100:1 with the proviso that when the cannabinoid:THC-type cannabinoid (CBC:THC) ratio is 1.1:0.9 and cannabidiol (CBD) is present in the composition, the ratio of CBD:CBC:THC is not 20:1.1:0.9 (CBD:CBC:THC). In still other aspects, the composition further comprises a CBC-type cannabinoid:THC-type cannabinoid ratio (CBC:THC). of from 1:2 to 100:1. In still yet further aspects, the CBC-type cannabinoid:THC-type cannabinoid ratio (CBC:THC) is from 1:3 to 20:1. In still yet further aspects, the CBC-type cannabinoid:THC-type cannabinoid ratio (CBC:THC) is from 1:4 to 15:1. In still yet further aspects, the CBC-type cannabinoid:THC-type cannabinoid ratio is from 1:5 to 10:1. In still yet further aspects, the CBC-type cannabinoid:THC-type cannabinoid ratio is from 1:6 to 5:1. In still yet further aspects, the CBC-type cannabinoid:THC-type cannabinoid ratio (CBC:THC) is from 1:7 to 4:1.

In yet still other aspects, the CBC-type cannabinoid: THC-type cannabinoid ratio (CBC:THC) is about 20:1. In yet still other aspects, the CBC-type cannabinoid: THC-type cannabinoid ratio (CBC:THC) is about 19:1. In yet still other aspects, the CBC-type cannabinoid: THC-type cannabinoid ratio (CBC:THC) is about 18:1. In yet still other aspects, the CBC-type cannabinoid: THC-type cannabinoid ratio (CBC:THC) is about 17:1. In yet still other aspects, the CBC-type cannabinoid: THC-type cannabinoid ratio (CBC:THC) is about 16:1. In yet still other aspects, the CBC-type cannabinoid: THC-type cannabinoid ratio (CBC:THC) is about 15:1. In yet still other aspects, the CBC-type cannabinoid: THC-type cannabinoid ratio (CBC:THC) is about 14:1. In yet still other aspects, the CBC-type cannabinoid: THC-type cannabinoid ratio (CBC:THC) is about 13:1. In yet still other aspects, the CBC-type cannabinoid: THC-type cannabinoid ratio (CBC:THC) is about 12:1. In yet still other aspects, the CBC-type cannabinoid: THC-type cannabinoid ratio (CBC:THC) is about 11:1. In yet still other aspects, the CBC-type cannabinoid: THC-type cannabinoid ratio (CBC:THC) is about 10:1. In yet still other aspects, the CBC-type cannabinoid: THC-type cannabinoid ratio (CBC:THC) is about 9:1. In yet still other aspects, the CBC-type cannabinoid: THC-type cannabinoid ratio (CBC:THC) is about 8:1. In yet still other aspects, the CBC-type cannabinoid: THC-type cannabinoid ratio (CBC:THC) is about 7:1. In yet still other aspects, the CBC-type cannabinoid: THC-type cannabinoid ratio (CBC:THC) is about 6:1. In yet still other aspects, the CBC-type cannabinoid: THC-type cannabinoid ratio (CBC:THC) is about 5:1. In yet still other aspects, the CBC-type cannabinoid: THC-type cannabinoid ratio (CBC:THC) is about 4:1. In yet still other aspects, the CBC-type cannabinoid: THC-type cannabinoid ratio (CBC:THC) is about 3:1. In yet still other aspects, the CBC-type cannabinoid: THC-type cannabinoid ratio (CBC:THC) is about 2:1. In still yet other aspects, the CBC-type cannabinoid:THC-type cannabinoid ratio (CBC:THC) is about 1:2. In still yet other aspects, the CBC-type cannabinoid:THC-type cannabinoid ratio (CBC:THC) is about 1:3. In still yet other aspects, the CBC-type cannabinoid:THC-type cannabinoid ratio (CBC:THC) is about 1:4. In still yet other aspects, the CBC-type cannabinoid:THC-type cannabinoid ratio (CBC:THC) is about 1:5. In still yet other aspects, the CBC-type cannabinoid:THC-type cannabinoid ratio (CBC:THC) is about 1:6. In still yet other aspects, the CBC-type cannabinoid:THC-type cannabinoid ratio (CBC:THC) is about 1:7 In still yet other aspects, the CBC-type cannabinoid:THC-type cannabinoid ratio (CBC:THC) is about 1:8. In still yet other aspects, the CBC-type cannabinoid:THC-type cannabinoid ratio (CBC:THC) is about 1:9. In still yet other aspects, the CBC-type cannabinoid:THC-type cannabinoid ratio (CBC:THC) is about 1:10. In still yet other aspects, the CBC-type cannabinoid:THC-type cannabinoid ratio (CBC:THC) is about 1:11. In still yet other aspects, the CBC-type cannabinoid:THC-type cannabinoid ratio (CBC:THC) is about 1:12. In still yet other aspects, the CBC-type cannabinoid:THC-type cannabinoid ratio (CBC:THC) is about 1:13. In still yet other aspects, the CBC-type cannabinoid:THC-type cannabinoid ratio (CBC:THC) is about 1:14. In still yet other aspects, the CBC-type cannabinoid:THC-type cannabinoid ratio (CBC:THC) is about 1:15. In still yet other aspects, the CBC-type cannabinoid:THC-type cannabinoid ratio (CBC:THC) is about 1:16. In still yet other aspects, the CBC-type cannabinoid:THC-type cannabinoid ratio (CBC:THC) is about 1:17. In still yet other aspects, the CBC-type cannabinoid:THC-type cannabinoid ratio (CBC:THC) is about 1:18. In still yet other aspects, the CBC-type cannabinoid:THC-type cannabinoid ratio (CBC:THC) is about 1:19. In still yet other aspects, the CBC-type cannabinoid:THC-type cannabinoid ratio (CBC:THC) is about 1:20.

In some embodiments, the composition includes from about 4% to about 99% of the CBC-type cannabinoid and/or from about 1% to about 96% of the THC-type cannabinoid by weight of total cannabinoids. In some embodiments, the composition includes from about 10% to about 85% CBC and from about 15% to about 90% THC; or from about 20% to about 85% CBC and from about 15% to 80% THC. In some embodiments, the composition includes from about 15% to about 30% THC and from about 70% to about 85% CBC; or from about 20% to about 25% THC and from about 75% to 80% CBC, where in some of these embodiments, the composition is a cannabis product for oral administration. In some embodiments, the composition includes from about 10% to about 20% CBC and from about 80% to about 90% THC, where in some of these embodiments, the composition is a cannabis product for oral administration.

In some embodiments, the composition includes from about 0.02 mg to about 5,000 mg of a CBC-type cannabinoid and from about 0.5 mg to about 5,000 mg by weight of a THC-type cannabinoid. In some embodiments, the composition includes from about 0.04 mg to about 5,000 mg of CBC and from about 1 mg to about 5,000 mg by weight of the THC. In yet other embodiments, the composition includes from about 0.02 mg to about 4,000 mg of a CBC-type cannabinoid and from about 0. 5 mg to about 4,000 mg by weight of a THC-type cannabinoid. In some embodiments, the composition includes from about 0.04 mg to about 4,000 mg of CBC and from about 1 mg to about 4,000 mg by weight of the THC. In some embodiments, the composition includes from about 0.02 mg to about 3,000 mg of a CBC-type cannabinoid and from about 0. 5 mg to about 3,000 mg by weight of a THC-type cannabinoid. In some embodiments, the composition includes from about 0.04 mg to about 3,000 mg of CBC and from about 1 mg to about 3,000 mg by weight of the THC. In some embodiments, the composition includes from about 0.02 mg to about 3,000 mg of a CBC-type cannabinoid and from about 0. 5 mg to about 3,000 mg by weight of a THC-type cannabinoid. In some embodiments, the composition includes from about 0.04 mg to about 3,000 mg of CBC and from about 1 mg to about 3,000 mg by weight of the THC. In some embodiments, the composition includes from about 0.02 mg to about 2,000 mg of a CBC-type cannabinoid and from about 0. 5 mg to about 2,000 mg by weight of a THC-type cannabinoid. In some embodiments, the composition includes from about 0.04 mg to about 2,000 mg of CBC and from about 1 mg to about 2,000 mg by weight of the THC. In some embodiments, the composition includes from about 0.02 mg to about 1,000 mg of a CBC-type cannabinoid and from about 0. 5 mg to about 1,000 mg by weight of a THC-type cannabinoid. In some embodiments, the composition includes from about 0.04 mg to about 1,000 mg of CBC and from about 1 mg to about 1,000 mg by weight of the THC. In some embodiments, the composition includes, from about 5 mg to about 500 mg by weight of CBC and from 5 mg to about 1000 mg by weight of THC.

In some embodiments, the composition includes cannabicyclol (CBL), cannabicitran (CBT), sesqui-CBC, sesqui-CBG, cannabidiol (CBD), cannabigerol (CBG), or any combinations thereof.

In some embodiments, the THC-type cannabinoid includes tetrahydrocannabinol (THC), tetrahydrocannabivarinol (THCV), tetrahydrocannabibutol (THCB), tetrahydrocannabihexol (THCH), tetrahydrocannabiphorol (THCP), hexahydrocannabinol (HHC), cannabinol (CBN), or any combinations thereof. In some embodiments, the THC-type cannabinoid includes THC. In some embodiments, the THC includes delta-8 THC, delta-9 THC, or a combination thereof. In some embodiments, the THC comprises delta-9 THC.

In some embodiments, the CBC-type cannabinoid comprises cannabichromevarin (CBCV), cannabichromene (CBC), cannabichromephorol (CBCP), cannabichromebutol (CBCB), cannabichromehexol (CBCH), sesqui-CBC, sesqui-CBCV, sesqui-CBCB, sesqui-CBCH, sesqui-CBCP, or any combinations thereof.

### Use of CBC-Type Cannabinoids to Modulate THC-Type Cannabinoids

According to an aspect, there is provided a use of CBC-type cannabinoids as described above in modulating the effects of a THC-type cannabinoid when administered to or consumed by a subject.

According to an aspect, there is provided a use of one or more compositions as described previously herein for delivering a modulated effect of a THC-type cannabinoid when administered to or consumed by a subject. In some embodiments, the modulated effect is compared to a control composition(s) comprising the THC-type cannabinoid without the CBC-type cannabinoid, or when a subject is administered or consumes only a single composition containing a THC-type cannabinoid without any previous or subsequent administration of a composition containing a CBC-type cannabinoid.

Methods for Modulating the Effect of THC-Type Cannabinoids with CBC-Type Cannabinoids

According to an aspect, there is provided a method for modulating the effects of a THC-type cannabinoid using a CBC-type cannabinoid as described previously herein.

According to an aspect, there is provided a method for delivering a modulated effect of a THC-type cannabinoid by administering to a subject or having a subject consume one or more compositions as previously described herein. In some embodiments, the modulated effect is compared to a control composition comprising the THC-type cannabinoid without the CBC-type cannabinoid. In other embodiments, the modulated effect is compared to a subject administered a single composition containing a THC-type cannabinoid without any previous or subsequent administration or consumption of a composition containing a CBC-type cannabinoid.

### Cannabinoid Dosage Forms

According to an aspect, the compositions described herein can be one or more dosage forms that are suitable for administration to or consumption by a subject. For example, the dosage form can be a tablet, a liquid, a capsule, a film (e.g., an oral film), a powder, an aerosol, a gel, a cream, an ointment, or any combination thereof. In some aspects, the dosage form contains a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. In some aspects, pharmaceutically acceptable carriers include buffers, solvents, dispersion media, coatings, isotonic and absorption delaying agents, and the like, that are compatible with administration to a subject. In one aspect, suitable carriers include phosphate buffered saline at concentrations ranging from about 1 μg/mL to about 1000 μg/mL, and in some aspects, about 100 μg/mL to about 500 μg/mL. In another aspect, suitable carriers may also include physiological saline, bacteriostatic water, Cremophor EL™ (BASF, Parsippany, N.J.) or phosphate buffered saline (PBS), optionally in admixture with surfactants such as polysorbates. In yet another aspect, suitable carriers may also include a solvent or dispersion medium containing, for example, water, ethanol, polyol (for example, glycerol, propylene glycol, and liquid polyethylene glycol), and suitable mixtures thereof. The carrier should be stable under the conditions of manufacture and storage and should be preserved against microorganisms. The use of carriers for pharmaceutically active substances is known in the art. For example, See Remington's Pharmaceutical Sciences, 18^{th} ed. (Mack Publishing Company, 1990).

Dosage forms can be prepared using techniques known in the art based on the intended route of administration. Examples of routes of administration include oral (including, for example, by oral gavage), intravenous (IV), peritoneal, intradermal, inhalation, pulmonary, transdermal, topical, transmucosal, rectal administration, or any combinations thereof. In some aspects, the dosage form is to be administered to the subject orally or buccally. In other aspects, the dosage form is to be administered intravenously. In other aspects, the dosage form is administered peritoneally. In other aspects, the dosage form is to be administered intradermally. In still other aspects, the dosage form is administered via the pulmonary route or by inhalation. In yet other aspects, the dosage form is to be administered transdermally. In yet other aspects, the dosage form is to administered topically. In still other aspects, the dosage form is to be administered transmucosally. In yet other aspects, the dosage form is to be administered rectally. In some aspects, the oral administration involves the dosage form being chewed or swallowed by the subject. In other aspects, the oral administration involves the dosage form being administered buccally.

In some aspects, when the dosage form is formulated for oral administration, the composition can further comprises a taste masking agent. For example, in some aspects, when the dosage form is a tablet, capsule, liquid or film, the dosage form can further comprise a taste masking agent. Cannabinoids generally have an unpleasant taste (see, Lus, G., Cantello, R., Danni, M.C., Rini, A., Sarchielli, P., Tassinari, T. and Signoriello, E., 2018. Palatability and oral cavity tolerability of THC: CBD oromucosal spray and possible improvement measures in multiple sclerosis patients with resistant spasticity: a pilot study. Neurodegenerative Disease Management, 8(2), pp. 105-113). In dosage forms containing both CBC-type cannabinoids and THC-type cannabinoids, the total cannabinoid in the composition may be higher than a composition without the CBC-type cannabinoid. The addition of a taste masking agent improves user acceptability of such compositions. In some embodiments, the taste masking agents includes a flavor, sweetener, emulsifier, coatings, complexing agents, or a combination thereof.

In some other aspects, when the dosage form is formulated for pulmonary administration, the composition can further comprise flavors, carriers, or a combination thereof.

### Cannabinoid Products

According to an aspect, the compositions described herein can be one or more products that include a CBC-type cannabinoid, a THC-type cannabinoid, or a combination of a CBC-type cannabinoid and a THC-type cannabinoid.

Cannabis products can be found in a variety of product forms that can be used to deliver cannabinoids via different routes of administration, such as by inhalation, ingestion (including consumption), topical administration, intravenous injection, peritoneal injection, or the like. In some embodiments, the cannabis product is administered by inhalation, ingestion or topical administration. In some embodiments, the cannabis product is administered by inhalation or ingestion. In still further embodiments, the cannabis product is ingested (e.g., consumed) by a subject.

In some embodiments, the cannabis product is an inhalable cannabis product. In some embodiments, the inhalable cannabis product includes a dried cannabis product, liquid cannabis vaporization composition, solid cannabis vaporization composition, or a combination thereof.

Cannabis plants generally produce relatively high amounts of the cannabinoids THC and CBD, but relatively low amounts of other cannabinoids, such as CBG or CBD. These cannabinoids are typically found in higher concentrations in glandular structures of cannabis plants called trichomes. These trichomes are typically more prevalent in the female flowers of cannabis plants. In some embodiments, the dried cannabis product includes dried cannabis, preferably dried female cannabis inflorescence. In some embodiments, the dried cannabis is reduced in size, such as by chopping, trimming, milling, grinding, or a combination thereof, to produce reduced dried cannabis.

In some embodiments, the dried cannabis product includes enriched dried cannabis. In some embodiments, the enriched dried cannabis includes enriched dried female cannabis inflorescences. Methods for enriching dried cannabis are known by skilled artisans, and include admixing, infusing, enrobing, spraying or panning adscititious cannabinoids onto the dried cannabis. The adscititious cannabinoids can be in liquid or solid form, depending on the nature of the cannabinoid and whether they are in a natural state, adsorbed onto a support medium or are dissolved in a liquid carrier. An example of an enriched dried cannabis product includes “moon rocks” where a dried female inflorescence is coated with kief.

In some embodiments, reduced dried cannabis is enriched with adscititious cannabinoids. Enriching dried cannabis with decreased particle sizes (such as in a ground or milled cannabis) increases the homogeneity of the resulting enriched product. When heated to vaporize endogenous and/or adscititious cannabinoids from the dried cannabis, increased cannabinoid homogeneity helps maintain a more uniform experience such as by decreasing the likelihood that one “puff” will have different cannabinoid content than another. When cannabis users partake in cannabis inhalation in social situations, multiple users sometimes share a dried cannabis product. It is desirable to maintain the uniformity of experience so that the users are sharing in a more similar experience and dosages. In some embodiments, the adscititious cannabinoids are incorporated into a paper product that is rolled, or chopped and mixed with dried cannabis. In some embodiments, the dried cannabis is formed into a packing, such as within a joint, spliff or blunt, and adscititious cannabinoids are injected into an interior portion of the packing, applied to the exterior of the packing, or a combination thereof.

Additional methods for enriching dried cannabis include genetic engineering made to the cannabis plant to increase cannabinoid concentrations therein. For example, cannabis cells may be genetically engineered to alter its cannabinoid biosynthesis pathway, such as by insertion of homologous genes from other organisms, insertion of additional copies of cannabis genes, insertion of engineered enzymes for a particular part of the cannabinoid biosynthesis pathway, or genetic engineering of cannabis cells to mutate endogenous genes to increase activity or expression of corresponding cannabinoid biosynthesis pathway enzymes (See, for example, Carvalho, supra; WO2019/209885; WO2020/176547, WO2021/034848, WO2021/195520, WO2021/257915, WO2022/011175; WO2022/081615; and U.S. Ser. No. 10/934,554) and/or by modifying the expression of transcription factors such as MYB transcription factors that can increase trichome density (See, for example, WO2022/169839 and WO2019/147873). Modified cells can then be regenerated into a plant with increased cannabinoid concentrations.

Conventionally, the most popular format for dried cannabis products are joints, spliffs and blunts, particularly joints. A joint comprises dried cannabis, such as dried female cannabis inflorescences, which is reduced in size (such as by grinding or milling) and rolled in a wrapping paper into a cannabis cigarette. The joint optionally includes a filter at a downstream end. The filter may aid allow for combustion of more of the dried cannabis joint, such as by allowing a user to hold onto it until all of the dried cannabis is combusted without burning their hands, remove unwanted combusted material, and/or reduce harshness. A spliff is a variation of a joint wherein the reduced dried cannabis is mixed with tobacco. A blunt is a variation of a joint wherein the wrapping paper is replaced with a leaf wrap, such as tobacco leaf or palm leaf. In some embodiments where the cannabis product is a combusted dried cannabis product, the cannabis product is a joint, spliff, or blunt. In some embodiments, the cannabis product is a joint, spliff, or blunt. In some embodiments, the cannabis product is a joint.

In some embodiments, the dried cannabis is a vaporized dried cannabis product. When vaporizing dried cannabis, dried cannabis is heated to a temperature that volatilizes cannabinoids, but does not result in the combustion of dried cannabis. Devices for vaporization of dried cannabis include the Volcano™ and Crafty™ vaporizers from Storz and Bickel, PAX™ vaporizers from Pax Labs, and Arizer Solo™ vaporizers from Arizer Tech. These devices generally work by admixing an influent carrier (such as air) with dried cannabis to produce a cannabinoid-enriched carrier, where the influent carrier and/or a chamber or cartridge is heated to a temperature that volatilizes cannabinoids in the dried cannabis. A user can then inhale the cannabinoid-enriched carrier. Use of vaporized dried cannabis products may result in fewer combustion by-products being inhaled by a user as compared to combusted dried cannabis products, and is believed to reduce the risk of negative long-term health effects. In some embodiments, vaporized dried cannabis products include dried cannabis optionally contained within a cartridge or pod.

In some embodiments, the dried cannabis product includes enriched dried cannabis, adscititious cannabinoid infusion, or a combination thereof. Methods for adscititious cannabinoid infusion include the addition of kief onto a dried cannabis product other than the dried cannabis (such as onto a surface of a rolling paper), the injection of a cannabinoid “slug” into a joint, spraying liquid cannabinoids onto a rolling paper, or other methods known to those skilled in the art.

In some embodiments, the cannabis product includes a cannabinoid vaporizer composition. Cannabinoid vaporizer compositions are used in cannabis vaporizers (“vapes”) whereby cannabinoids in the cannabinoid vaporizer compositions are vaporized and/or aerosolized for inhalation. In some embodiments, vapes include “oil vapes”, dried powder inhalers, nebulizers, and “dab rigs”. In some embodiments, cannabinoid vaporizer compositions include a cannabinoid and optionally a carrier. In some embodiments, cannabinoid vaporizer compositions further include flavors. In some embodiments, cannabinoid vaporizer compositions include cannabis extracts (including full spectrum extracts, sap, wax, crumble, budder, and shatter), cannabis distillate, purified cannabinoid (including isolated plant cannabinoids, biosynthetic cannabinoids, and chemically synthesized cannabinoids), live rosin, live resin, or a combination thereof. In some embodiments, cannabinoid vaporizer compositions are included within a “cartomizer” cartridge, such as 510 cartridges from Shenzhen Smoore Technology Limited, Shenzhen Kanger Technology Co., Ltd., or Joyetech (Shenzhen) Electronics Co. Ltd., or Pax Era™ pods from PAX Labs, Inc., or within a disposable all-in-one device.

In some embodiments, cannabinoid vaporizer compositions are enriched with a target cannabinoid to obtain an enriched cannabinoid vaporizer compositions. In some embodiments, the enriched cannabinoid vaporizer compositions include adscititious cannabinoids, are extracted from biomass with increased levels of the target cannabinoid, or are extracted in a manner that preferentially enriches the extract with the target cannabinoid. As cannabinoid vaporizer compositions are generally in a liquid form, or processed such that there is a liquid intermediate, it may be possible to incorporate the adscititious cannabinoid within the vaporizer composition homogenously by admixing.

In some embodiments, the cannabis product is an ingestible cannabis product. In some embodiments, the ingestible cannabis product includes a cannabis edible, a beverage, a tincture, or a lozenge Ingestible cannabis products are a popular format for using cannabis because there is no odor associated with combustion, or, in some cases, vaporization, of cannabis material. This allows use to be done discretely and decreases the likelihood of annoying those in a user's vicinity.

In preparing an ingestible product, cannabis or cannabinoids must be incorporated into the edible product. The cannabis plant produces cannabinoids in their acidic form, such as delta-9 THCA. However, delta-9 THCA does not provide the “high” associated with cannabis, and must be converted into delta-9 THC by decarboxylation. When combusted, such as when smoking a joint, the heat of combustion will cause this reaction such that the user inhales THC in its decarboxylated form. However, the temperatures associated with ingestion of cannabis edibles (e.g. body temperature) do not result in decarboxylation. Cannabinoids are often decarboxylated prior to their incorporation into cannabis products, or during the manufacture of the cannabis product if the conditions, such as time and temperature, to sufficiently convert the THCA into THC. Further, although dried cannabis may be incorporated into cannabis edibles, the dried plant material may impart unpleasant textures and not be ideal for incorporation. As such, cannabis extracts, purified cannabis isolates, synthetically produced cannabinoids or biosynthetically produced cannabinoids may be used to provide the effects of the cannabinoids without the textural disadvantages of dried cannabis.

Additional considerations in the preparation of ingestible cannabis products are that cannabinoids are poorly soluble in water, have relatively low bioavailability and have relatively slow onset times. As such, in some embodiments, the ingestible cannabinoid product includes one or more excipients. Typically, in ingestible cannabis products, cannabinoids are incorporated into an oil phase solvent or oil-based ingredients, admixed with emulsifying agents, admixed with co-solvents, subject to chemical modifications (such as glycosylation or alkaline treatments) to increase water solubility, or a combination thereof. Some of these additions may result in the formation of emulsions, microemulsions, nanoemulsions, liposomes, or the like.

In some embodiments, the cannabis edible is a gummy, chocolate, confection, chewing gum, or baked good. Cannabis edibles are often incorporated into a sweet treat as the sweetness and flavors imparted by the sweet treat can improve the palatability of cannabinoids, many of which have a taste and smell that some users find unpleasant. However, depending on the total cannabinoids infused in the cannabis edible, these components may be insufficient to mask the taste of the cannabinoid. As such, in some embodiments, the cannabis edible includes a taste masking agent. In some embodiments, the taste masking agent includes a flavor, sweetener, emulsifier, coating, complexing agent, or a combination thereof.

In some embodiments, the cannabis edible is a confection, preferably a gummy. Historically, “pot brownies” were an especially popular form for cannabis edibles. However, due to its smaller size, leading to its ability the be ingested more discretely and ease of transport; its relative ease to control dosing by taking more pieces as opposed to dividing a brownie; and its longer shelf life, confections, such as gummies have become an increasingly popular form.

The following examples are not intended to be limiting.

### EXAMPLES

### Example 1—Preparation of Cannabinoid-Infused Gummies

A pectin-based gummy base was prepared. THC and CBC in a ratio of about 1:3 was dissolved in a medium chain triglyceride solvent and admixed with the gummy base to produce a cannabinoid-infused gummy base. The cannabinoid-infused gummy base was cooked and deposited as discrete pieces into molds, with each piece weighing five grams. The pieces were then allowed to cool to form cannabinoid-infused gummy and then coated with a sweet and sour sanding mixture to produce a finished gummy. The cannabinoid content of each gummy is set out in Table 1, below.

TABLE 1
Cannabinoid content of each gummy

| | mg of | | |
|---|---|---|---|
| | Cannabinoid | mg/g in | Rel. % of total |
| Cannabinoid Name | per gummy | gummy | cannabinoid |
| Δ9-THC | 2.000 | 0.400 | 23.56 |
| Δ9-THCA | 0.000 | 0.000 | 0.00 |
| Δ8-THC | 0.000 | 0.000 | 0.00 |
| Δ8-THCA | 0.000 | 0.000 | 0.00 |
| CBG | 0.099 | 0.020 | 1.17 |
| CBGA | 0.000 | 0.000 | 0.00 |
| CBD | 0.019 | 0.004 | 0.22 |
| CBDA | 0.000 | 0.000 | 0.00 |
| CBC | 6.066 | 1.213 | 71.47 |
| CBCA | 0.000 | 0.000 | 0.00 |
| CBL | 0.010 | 0.002 | 0.12 |
| CBT | 0.010 | 0.002 | 0.12 |
| Sesqui-CBC | 0.146 | 0.029 | 1.72 |
| Sesqui-CBG | 0.045 | 0.009 | 0.53 |
| CBN | 0.093 | 0.019 | 1.10 |

### Example 2—Perception Study

An organoleptic and experiential study was conducted to determine the potential market acceptance of a novel cannabinoid-infused gummy product. Twenty participants were recruited for this study. Inclusion criteria for the participants included age over 19 years, who had consumed THC-infused gummies at least once in the past 3 months in a dose of at least 2 mg THC per session and with no food allergies or other dietary restrictions. Participants provided written informed consent.

Participants were read a potential marketing description that references flavors and need state of the product, and then each given five gummies (prepared according to Example 1). The participants cleansed their palate with unsalted crackers and water prior to evaluating the gummies. The participants were then allowed to evaluate their gummies and then answered a questionnaire including overall liking, meeting expectations, and sensory characteristics. Follow-up questionnaires were sent approximately 2.5 hours and 24 hours after evaluation.

The participants scored an overall liability in a 9-point scale of 8.2 upon evaluation, and remained relatively unchanged at 8.1 at 24 hours following the evaluation.

In the follow-up questionnaires, participants indicated that the product was generally a balanced experience with a slight skewing to a more energized experience. Additionally, results generally showed participants indicating that they felt the product would be consistent with experiences associated with enjoying the moment and unwinding; laughing going outside and being social; and inconsistent with being focused, concentrating and working; zoning out; wanting to engage in active pursuits; or resting. These experiences tend to be associated with modulation of the high, for example, modulated moods.

### Example 3—Tetrad Study

### Materials and Methods and Compounds

THC (Cat. No. 12068) and (±)-CBC (Cat. No. 26252) were purchased from Cayman Chemical (Ann Arbor, MI). All certified reference standards and materials (including CBC, THC, 11-hydroxy-Δ⁹-tetrahydrocannabinol [11-OH-THC], and carboxy-Δ⁹-tetrahydrocannabinol [COOH-THC]) were purchased from Sigma Aldrich Research Chemicals (Oakville, ON) suspended in acetonitrile. The blends of CBC and THC, namely the blends of 1:6.92 (CBC:THC) and 3.90:1 (CBC:THC), respectively, were provided as a resin that was resuspended in treatment vehicles described below. All other reagents were obtained from Sigma-Aldrich unless specifically noted.

Animals Used in this Study

Female and male C57BL/6 mice between 8 and 12 weeks of age were used for these studies. Animals were group housed at the Laboratory Animal Services Unit (LASU) at the University of Saskatchewan (5 mice/cage for females, 3 mice/cage for males) with a standard 12:12 light-dark cycle, ad libitum access to food and water, and environmental enrichment. All compounds were prepared in a vehicle composed of ethanol and kolliphor in saline (1:1:8) for i.v. tail vein injections or olive oil for oral gavage (p.o.) at the doses indicated in description of the figures. Compounds were administered at the doses indicated in mg/kg body weight, with body weights being measured on the same day as drug administration. Experimenters were blinded to treatment for all assessments and analyses. Animals were purchased from Charles River Labs (Senneville, QC), rather than bred, to reduce animal numbers. At the end of experiments, mice were euthanized via isoflurane overdose and cardiac puncture. In all cases, experiments were performed with the approval of the University Animal Care Committee (UACC) at the University of Saskatchewan (Animal Use Protocol #20200043) and in keeping with the guidelines of the Canadian Council on Animal Care (CCAC).

For time course PK experiments, female and male mice were treated with 10 mg/kg compound (CBC, THC, 1:6.92 (CBC:THC), or 3.90:1 (CBC:THC)) i.v., or p.o. once. Two blood samples were obtained per animal using a destructive serial blood sampling approach, such that the first sample was a saphenous vein blood collection of 30 μL and the second sample was collected by cardiac puncture following euthanasia. Sampling timepoints were 10 min, 1 h, 6 h, and 12 h post-compound administration. Blood samples were subjected to high performance liquid chromatography tandem mass spectrometry (HPLC-MS/MS) to quantify compound and compound metabolite levels. These data were used to determine Cₘₐₓ, tₘₐₓ, and t_{1/2} for each tested compound and assess potential differences exist between CBC, THC, 1:6.92 (CBC:THC) and 3.90:1 (CBC:THC).

### Physiological Assessment

Mice were assessed in the ‘tetrad’ battery of physiological and behavioural assessments. This included, in the following order: a bar holding assessment of catalepsy, rectal thermometer assessment of body temperature, a test of nociception in the warm water tail flick assay, and a test of locomotion and anxiety in the open field test (OFT). The precise timing of each measurement are stated in description of the figures. The same animals were used for all four measures of the tetrad.

Catalepsy was assessed in the bar holding assay after compound administration with mice placed so that their forepaws clasped a 0.7-cm ring clamp 4.5 cm above the surface of the testing space. The length of time the ring was held was recorded up to 60 sec (i.e., percent maximum possible effect [MPE] 60 seconds) with the trial ending if the mouse turned its heondsad or body or made three consecutive escape attempts. Activation of CB1R is associated with increased catalepsy.

Body temperature was measured after compound administration by using a rectal thermometer. Activation of CB1R is associated with reduced body temperature.

Anti-nociceptive effects were measured in warm water (52±2° C.) using the tail-flick test after compound administration to a maximum of 10 seconds (i.e., percent maximum possible effect [MPE]10 sec). Activation of CB1R is associated with elevated tail-flick latency (i.e. less nociception as indicated by the mouse keeping their tail in the water longer).

Locomotion was measured in the open field test (OFT) after compound administration. Mice were placed in the centre of an open chamber measuring 1 m×1 m for 5 minutes. Total distance travelled and time spent in the central quadrant of the field are recorded on a video camera, and the video analyzed in EthoVision XT (Noldus Information Technology Inc., Leesburg, VA).

### Behavioural Assessment

In addition to the physiological assessment of the tetrad, mice were subjected to several behavioural assessments detailed here. These experiments followed a set timeline beginning 30 minutes after compound administration, based on an approximation that the maximum blood concentration of drug occurred 30-60 minutes after animals had received oral gavage. In these experiments the same type of mice were used for elevated plus maze, y-maze, and forced swim test back-to-back-to-back; however, this was a separate cohort of mice from those used in tetrad or PK experiments.

Modelled anxiety—the elevated plus maze: Mice were placed in the centre space of an elevated plus maze (EPM) standing 45 cm above the floor and having 2 closed-walled arms and 2 open arms each measuring 45 cm long. Mice were placed facing an open arm and their movement recorded for 5 minutes beginning 30 minutes after drug administration. Total distance travelled, arm entries, and time spent in each arm was recorded on a video camera and scored by an individual blinded to treatment group.

Modelled exploratory behaviour and cognition—the y-maze: The y-maze consists of three 45 3 cm long×15 cm wide arms equally spaced 120° apart. Mice were allowed to habituate to the y-maze with one arm closed off (the novel arm) for 15 minutes 1 day prior to the experiment. On testing day, mice were placed in the centre space the y-maze facing the novel arm. Movement and arm entries were recorded for 5 min beginning 40 minutes after drug administration. Total distance travelled, arm entries, and % correct alternations between each arm were recorded on a video camera and scored by an individual blinded to treatment group.

Modelled behavioural despair and resiliency—the forced swim test: Mice were placed in 800 mL of 22° C. water in a clear beaker with a 10 cm diameter such that the depth of water was approximately 10-11 cm. Mice were allowed to swim and their movement recorded for 5 minutes beginning 50 minutes after drug administration. Time spent swimming and time spent immobile was recorded on a video camera and scored by an individual blinded to treatment group.

Pharmacokinetic assessment—high performance liquid chromatography tandem mass spectroscopy (HPLC-MS/MS)

Whole blood was collected from mice at designated time points as indicated in the description of the figures and stored in 5 mL LoBind Eppendorf tubes (Cat. No. 0030122356), frozen in liquid nitrogen and stored at −80° C. Cardiac blood was collected by syringe and immediately transferred to 4 mL BD Vacutainer tube (Cat. No. CABD367884). Whole blood was collected, aliquoted at 30 μL into 1.5 mL Lobind Eppendorf tubes (Cat. No.0030108442), frozen, and stored at −80° C. until needed.

The quantification of blood concentrations of CBC, THC, and THC's metabolites 11-OH-THC and COOH-THC was performed using HPLC-MS/MS (adapted from Roebuck, A J, Greba, Q, Onofrychuk, T J, McElroy D L, Sandini T M, Zagzoog A, et al. (2022). Dissociable changes in spike and wave discharges following exposure to injected cannabinoids and smoked cannabis in Genetic Absence Epilepsy Rats from Strasbourg. Eur. J. Neurosci. 55: 1063-1078). Phytocannabinoids were quantified using Agilent 1260 binary pump LC system (Agilent Technologies Canada, Mississauga, ON) coupled to an ABSciex 6500QTRAP (ABSciex, Concord, ON) mass spectrometer using an Eclipse Plus phenyl hexyl column (4.6 Å 100 mm, 5 μm column, Agilent) and 1290 Infinity II inline filter (0.3 μm). HPLC-MS/MS was conducted and peaks analyzed using Analyst (version 1.7). Cerriliant (Sigma-Aldrich) internal standards for deuterated (d3)-CBC, CBC, THC, THC-deuterated (d3) 11-OH-THC, and COOH-THC were diluted to 1 mg/mL (if not originally provided at this concentration) and stored at −20° C. Standard curves were established between 3.91 ng/mL and 5,000 ng/mL. The lower limit of quantification (LLOQ) for both CBC and THC was 1.97 ng/mL. For 11-OH-THC and COOH-THC the LLOQ was 10 ng/mL. The upper limit of quantification (ULOQ) was set as 4,000 ng/mL for all compounds. All phytocannabinoids and their metabolites shared a medium quality control (MQC) of 100 ng/mL and a high-quality control (HQC) of 175 ng/mL. Standard curve and quality controls were prepared by mixing 10 μL of standard dilutions to 190 μL of blank mouse whole blood. Six hundred μL of prepared internal standard solution at 1.84 pg/mL was added to all standards and QCs, vortexed and centrifuged at 14,000×g at 4° C. to precipitate proteins. Supernatant was collected, vacuum filtered through an Agilent Captiva EMR-Lipid 96-well plate (Cat. No. 5190-1001) and transferred to an amber autosampler vial for testing. To prepare non-blood tissues, samples were thawed at room temperature, 600 μL of internal standard solution was added, samples were vortexed, centrifuged and filtered as above.

### Statistical Analysis

Data analyses were performed by an individual blinded to treatment groups. All analyses were completed in GraphPad Prism (v. 9.2, La Jolla, CA). PK data were assessed by a simple area under the curve (AUC) analysis to determine Cₘₐₓ, tₘₐₓ, and AUC_{0-∞}. Bioavailability (F) was calculated as the mean AUC_{0-∞}0 for a route of administration divided by the mean AUC_{0-∞} for i.v. administration. Apparent elimination half-life (t₁/2) was calculated using a one phase exponential decay model with the minimum constrained to 0. Dose-response data were fitted to a three parameter non-linear regression analysis to estimate potency (ED₅₀) and efficacy (Eₘₐₓ). Data were expressed as the mean±SEM. Statistical analyses were conducted by one-way analysis of variance (ANOVA). Post-hoc analyses were performed using Dunnett's test. All results are reported as the mean±the standard error of the mean (SEM). p values <0.05 were considered to be significant.

### Results

### Physiological Assessment

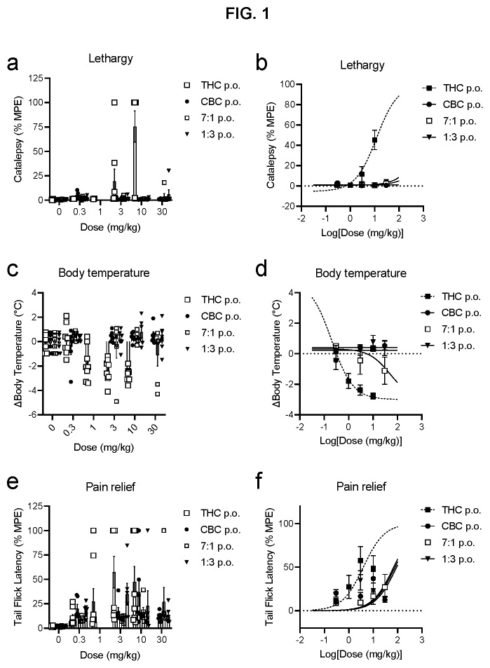

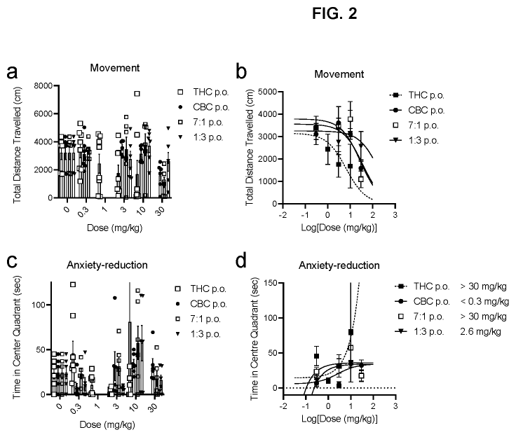

The first assessment of the CBC compositions was in the mouse tetrad, which consisted of catalepsy as a model of rigidity or lethargy, change body temperature, the tail flick nociception test as a model of pain perception or ‘pain relief’, and movement in the OFT to assess overall movement ability and as a rodent model of anxiety based on the time mice spent away from the walls of the OFT and in the center of the test space (FIGS. 1 and 2, Table 1 and 2). For all of the tests described below male and female mice were used but no sex differences were observed and therefore data are presented with male and female datasets combined.

Catalepsy—Catalepsy was tested in the ring holding assay 5 minutes after compound treatment. As expected, mice treated with THC experienced increasing catalepsy in a dose-dependent manner with a potency of approximately 12 mg/kg and a maximum effect of 76% (100% being 60 seconds without movement) (FIG. 1A, FIG. 1B; Table 1). In contrast, CBC, 1:6.92 (CBC:THC), and 3.90:1 (CBC:THC) produced no dose-dependent catalepsy. These data demonstrate that when CBC is present alongside THC—as in the case of both 1:6.92 (CBC:THC) and 3.90:1 (CBC:THC) compounds—the lethargy induced by THC was eliminated.

Body temperature—Hypothermia is a hallmark effect of cannabinoids in rodent models and is thought to occur through activation of CB1R in the hypothalamus (Grim T W, Morales A J, Thomas B F, Wiley J L, Endres G W, Negus S S, Lichtman A H (2017). Apparent CB₁ Receptor Rimonabant Affinity Estimates: Combination with THC and Synthetic Cannabinoids in the Mouse In Vivo Triad Model. J. Pharmacol. Exp. Ther. 362: 210-218). Although hypothermia is not commonly described in human users of cannabis, the hypothalamus is a key center for the regulation of metabolic processes in humans and CB1R is abundant in this region of the human brain (Ripamonte G C, Bernardes-Ribeiro M, Patrone L G A, Vicente M C, Bicego K C, Gargaglioni L H (2020). Functional role for preoptic CB1 receptors in breathing and thermal control. Neurosci. Lett. 732: 135021). Body temperature was measured prior to compound administration and 10 min after compound administration. As expected, mice treated with THC experienced dose-dependent hypothermia with a potency of approximately 0.2 mg/kg and a maximum effect of −2.8° C. change in body temperature (FIG. 1C, FIG. 1D; Table 1). In contrast, CBC and 3.90:1 (CBC:THC) produced no dose-dependent hypothermia. The 1:6.92 (CBC:THC) product that contains more THC and CBC produced some hypothermia (−1.1° C.) but the potency extended beyond the range of doses tested. These data demonstrate that when CBC is present alongside THC—as in the case of both 1:6.92 (CBC:THC) and 3.90:1 (CBC:THC) compounds—hypothermic effects were either greatly reduced (1:6.92 (CBC:THC)) or eliminated.

Nociception (i.e., pain perception)—Cannabinoids are known to reduce pain sensation through several mechanisms including CB1R, transient receptor potential channels (TRPs), and inhibiting inflammation (reviewed in Turner S E, Williams C M, Iversen L, Whalley B J (2017). Molecular Pharmacology of Phytocannabinoids. Prog. Chem. Org. Nat. Prod. 103: 61-101]. Pain perception was measured in the warm water tail flick test 20 minutes after compound administration. As expected, mice treated with THC experienced dose-dependent reduction in pain sensation with a potency of approximately 3.8 mg/kg and a maximum effect of 57% (100% being 20 seconds of sustained presence of the animal's tail in warm water) (FIG. 1E, FIG. 1F; Table 1). CBC, 3.90:1 (CBC:THC), and 1:6.92 (CBC:THC) all also produced dose-dependent pain-relieving effects albeit with lower potency and with efficacy that was lower than that of THC but not statistically different. Of note, the 3.90:1 (CBC:THC) product appeared to produce a bell-shaped dose-response that actually peaked at 3 mg/kg and then fall in efficacy at higher doses. The reason for this is bell-shaped response is unclear. These data demonstrate THC and CBC reduce pain sensation in mice and when used in combination the effect is more similar to that of CBC, but still readily observed and significant. Therefore, both 1:6.92 (CBC:THC) and 3.90:1 (CBC:THC) appear to be useful in the treatment of some types of pain.

Open field test—Cannabinoid agonists of CB1R reduce movement in the OFT and increase the time a mouse spends in the center of the OFT, which is a model test for anxiety-relieving effects (Grim T W, Morales A J, Thomas B F, Wiley J L, Endres G W, Negus S S, Lichtman A H (2017). Apparent CB₁ Receptor Rimonabant Affinity Estimates: Combination with THC and Synthetic Cannabinoids in the Mouse In Vivo Triad Model. J. Pharmacol. Exp. Ther. 362: 210-218; Wiley J L, Martin B R (2003). Cannabinoid pharmacological properties common to other centrally acting drugs. Eur. J. Pharmacol. 471: 185-193). Movement in the OFT was measured 30 minutes after compound administration. Mice treated with THC experienced dose-dependent reduction in movement with a potency of approximately 6.6 mg/kg and a maximum effect of reducing movement to 1,700 cm from the vehicle-treated level of approximately 3,300 cm in 5 minutes (i.e., a 48% reduction in movement) (FIG. 2A, FIG. 2E; Table 2). CBC, 3.90:1 (CBC:THC), and 1:6.92 (CBC:THC) all also produced dose-dependent reduction in movement that was actually greater in efficacy for CBC and 1:6.92 (CBC:THC) than THC, although not statistically significant and with lower potency than THC for CBC and 3.90:1 (CBC:THC). Similar to pain perception, these data demonstrate THC and CBC reduce movement in mice and when used in combination the effect is more similar to that of CBC, but still readily observed and significant. Therefore, both 1:6.92 (CBC:THC) and 3.90:1 (CBC:THC) reduce movement overall.

Similar to overall movement, mice treated with THC experienced a dose-dependent increase in the total time spent in the center of the OFT although the potency was not within the range of doses tested here (FIG. 2C, FIG. 2D; Table 2). This reduced potency of THC compared to earlier observations is likely due to the change in route of administration from injection to oral gavage (Zagzoog A, Mohamed K A, Kim H J J, Kim E D, Frank C S, et al. (2020). In vitro and in vivo pharmacological activity of minor cannabinoids isolated from Cannabis sativa. Sci. Rep. 10: 20405), CBC, 3.90:1 (CBC:THC), and 1:6.92 (CBC:THC) all also produced an anxiety-reducing effect as shown by more time spent in the center of the OFT; however the size of this effect was lower than that of THC. These data are important because it demonstrates THC and CBC are anxiety-reducing (i.e., anxiolytic) in mice and when used in combination the effect is not different compared to either drug alone. In fact, the data seem more consistent and less variable for animals receiving 1:6.92 (CBC:THC) or 3.90:1 (CBC:THC) for the anxiolytic effects than THC alone. Therefore, both 1:6.92 (CBC:THC) and 3.90:1 (CBC:THC) may have anxiety reducing effects in the OFT test.

### Behavioural Assessment

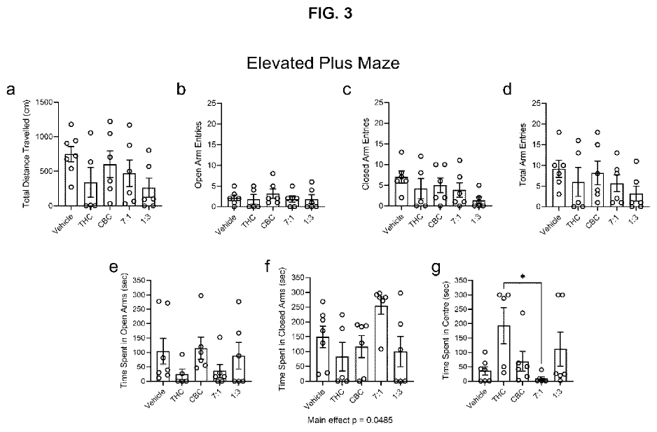

Modelled anxiety—the elevated plus maze (EPM)—Following up on the findings in the OFT we next assessed anxiety-like behaviour in the EPM. Mice were treated with a single 10 mg/kg p.o. dose of the indicated compound and then assessed 30 minutes after treatment (FIG. 3). Similar to the observations in the OFT, 10 mg/kg THC reduced overall movement in the EPM, although this change was not statistically significant; likewise a non-significant trend toward locomotor depression was observed for both 1:6.92 (CBC:THC) and 1:3 (CHB:THC; FIG. 3A). We next compared arm entries into the open (FIG. 3B) and closed arms of the EPM (FIG. 3C). Although there was a trend toward CBC producing more open arm entries than other groups, this was not statistically significant (FIG. 3B). No differences were observed for closed arm entries (FIG. 3C). Total arm entries—simply the sum of open and closed arm entries—followed the same trend as total movement in the EPM and no significant differences were detected (FIG. 3D). The time spent in each arm and the center of the EPM (FIG. 3E- FIG. 3G) was measured. THC and 1:6.92 (CBC:THC) appeared to suppress time in the open arms—which would be indicative of an anxiety-producing effect—but these differences were not statistically significant (FIG. 3E). A main effect of treatment was detected in the one-way ANOVA for time spent in the closed arms (p=0.0485), but post-hoc testing did not detect differences between treatment groups (FIG. 3F); despite this, a trend toward less time spent in the closed arms was observed for THC, CBC, and 3.90:1 (CBC:THC) and more time spent in the closed arms for 1:6.92 (CBC:THC; FIG. 3G). The center of the EPM is where the mice begin their testing and this space is open; therefore, time in the center of the EPM can be considered as an anxiety-reducing effect or the consequence of locomotor suppression because the animal simply does not leave the central space. Because of this, the time in the center is analyzed separately. THC treatment produced more time in the center compared to 1:6.92 (CBC:THC) and this was likely due to locomotor suppression in the THC-treated animals (FIG. 3F). A trend toward more time in the center was observed for the 3.90:1 (CBC:THC) compound, which is believed to be indicative of an anxiety-reducing effect (FIG. 3F).

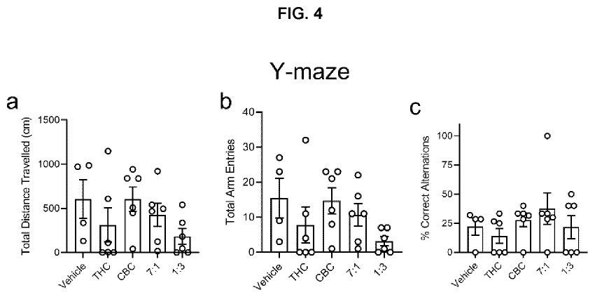

Modelled exploratory behaviour and cognition—the y-maze—After testing mice in the EPM, cognition and working memory was assessed in the y-maze. Changes in cognition and working memory—and by extension curiosity—are indicated by changes in % correct alternations between the three arms of the y-maze. Y-maze analyses were conducted 40 min after compound administration in the same mice that had been used for EPM testing. Similar to the OFT and EPM, THC treatment was associated with reduced overall locomotion in the y-maze (FIG. 4A). A similar trend toward reduced locomotion was also observed for 1:6.92 (CBC:THC) and 3.90:1 (CBC:THC) treatments (FIG. 4A). Likewise, total arm entries were lower in mice treated with THC or 3.90:1 (CBC:THC), but not 1:6.92 (CBC:THC) or CBC (FIG. 4B). TA trend was present where THC appeared to reduce correct alternations between y-maze arms (FIG. 4C). 3.90:1 (CBC:THC) was not different from vehicle treatment (FIG. 4C). Importantly, 1:6.92 (CBC:THC; and to a lesser extent CBC) increased % correct alternations compared to vehicle. These data suggest that although pure THC may reduce working memory, such deficits are not present when THC is combined with CBC in 1:6.92 (CBC:THC) and 3.90:1 (CBC:THC). Moreover, 1:6.92 (CBC:THC) may improve cognition and working memory.

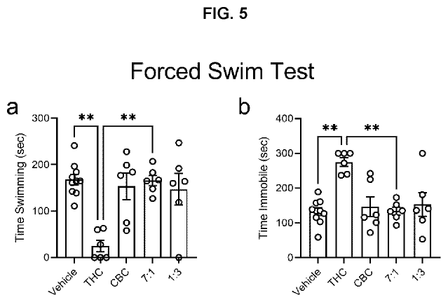

Modelled behavioural despair and resiliency—the forced swim test (FST)—Following the y-maze, resiliency was assessed through the FST. In this assay, increased time spent swimming rather than immobile is thought to model mood enhancement, including anti-depressant or resiliency-increasing effects. Treatment of mice with the vehicle itself has been shown to reduce swimming and therefore comparisons here are made against the THC treatment. Even compared to vehicle, THC further suppressed swimming time and increased immobile time (p=0.0049) (FIG. 5). In contrast, CBC, 1:6.92 (CBC:THC), and 3.90:1 (CBC:THC) all had similar time spent swimming as vehicle (FIG. 5); and 1:6.92 (CBC:THC) significantly increased time swimming and decreased immobile time compared to THC (p=0.0038). Therefore, the presence of even small amounts of CBC with THC—such as in 1:6.92 (CBC:THC)—is sufficient to reverse THC's anti-resiliency effects. These data indicate 1:6.92 (CBC:THC) and 3.90:1 (CBC:THC) improved resiliency in the mouse FST compared to THC, especially and significantly in the case of 1:6.92 (CBC:THC).

Pharmacokinetic assessment—high performance liquid chromatography tandem mass spectroscopy (HPLC-MS/MS)—In order to understand how THC and CBC absorption, metabolism, and elimination were altered by one another mice were treated with 10 mg/kg i.v. or p.o. of each compound and blood samples were taken 10 minutes, 1 hour, 6 hours, and 12 hours post-compound administration. Data are summarized here for each compound measured: CBC, THC, and THC's two primary metabolites 11-OH-THC and COOH-THC.

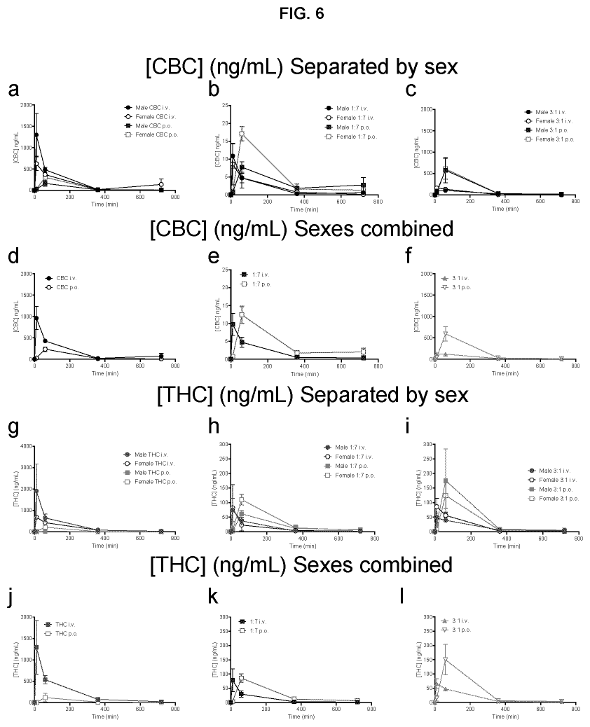

For CBC measurements, female mice metabolized i.v. CBC slower than males, but this difference in metabolism was reversed or absent in female mice treated with 1:6.92 (CBC:THC) or 3.90:1 (CBC:THC) FIG. 6A-6f, Table 3). The combination of THC+CBC in both 1:6.92 (CBC:THC) and 3.90:1 (CBC:THC) significantly slowed the metabolism of CBC for i.v. and p.o. groups (FIG. 6A-6C, Table 3). Therefore, more CBC was present for more time in these animals. The combination of THC+CBC in both 1:6.92 (CBC:THC) and 3.90:1 (CBC:THC) had a large effect on bioavailability—improving CBC uptake in the body approximately 8-fold compared to CBC alone.

For THC measurements, female mice also metabolized i. v. THC slower than males (as observed for CBC), but this difference in metabolism was absent in female mice treated with 7:1 or 1:3 (FIG. 6G-6L, Table 4). The combination of THC+CBC in both 1:6.92 (CBC:THC) and 3.90:1 (CBC:THC) significantly sped up the metabolism and/or elimination of THC for i.v. and p.o. groups. Therefore, less THC was present for less time in these animals. The combination of THC+CBC in both 1:6.92 (CBC:THC) and 3.90:1 (CBC:THC) had a large effect on bioavailability—improving THC uptake in the body approximately 14-fold compared to THC alone.

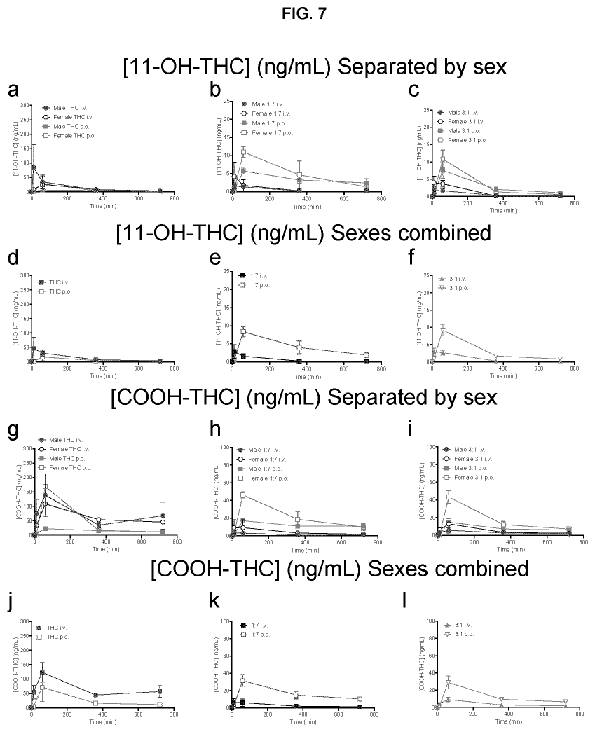

For the metabolism of THC into its main metabolites 11-OH-THC and COOH-THC, female mice also metabolized i.v. 11-OH-THC slower than males, but this difference in metabolism was reversed in female mice treated with 1:6.92 (CBC:THC) or 3.90:1 (CBC:THC; FIG. 7, FIG. 8; Tables 5, 6). The combination of THC+CBC in 3.90:1 (CBC:THC) significantly sped up the metabolism of 11-OH-THC for i.v. and p.o. females. Combination treatments also sped up metabolism of 11-OH-THC in 1:6.92 (CBC:THC) i.v. females. This means that less 11-OH-THC was present for less time in these animals receiving both 1:6.92 (CBC:THC) and 3.90:1 (CBC:THC). No major changes were noted for other groups for this metabolite. The combination of THC+CBC in both 1:6.92 (CBC:THC) and 3.90:1 (CBC:THC) had a large effect on bioavailability—improving THC uptake in the body approximately 10-fold compared to THC alone. For the metabolism of THC into its main metabolites 11-OH-THC and COOH-THC: no sex differences in metabolic rate were noted. The combination of THC+CBC in 3.90:1 (CBC:THC) or 1:6.92 (CBC:THC) significantly sped up the metabolism of COOH-THC for i.v. and p.o. mice. Meaning less COOH-THC was present for less time in these animals. As with 11-OH-THC, the combination of THC+CBC in both 1:6.92 (CBC:THC) and 3.90:1 (CBC:THC) had a large effect on bioavailability—improving THC uptake in the body approximately 10-fold compared to THC alone.

From all of these PK data it appears that from a drug kinetics stand point, the combinations of THC+CBC in the 3.90:1 (CBC:THC) and 1:6.92 (CBC:THC) products slow CBC metabolism, speed up THC metabolism, and increase bioavailability of both drugs. Based on these data it is predicted that the combination treatments of 3.90:1 (CBC:THC) or 1:6.92 (CBC:THC) would produce less intoxication than THC alone and that the overall effect would resemble that of CBC. That PK observation is consistent with all of the behavioural and physiological data.

TABLE 2
Assessment of mouse physiology following cannabinoid oral gavage.

| | Lethargy | | Body temperature | | Pain relief | |
|---|---|---|---|---|---|---|
| | ED₅₀ (95% | | ED₅₀ (95% | Eₘₐₓ ± S.E.M. | ED₅₀ (95% | |
| Compound | C.I.) (mg/kg) | Eₘₐₓ ± S.E.M. (%) | C.I.) (mg/kg) | (° C.) | C.I.) (mg/kg) | Eₘₐₓ ± S.E.M. (%) |
| THC | 12 (7.1-21) | 76 ± 16 | 0.2 (0.1-1.1) | −2.8 ± 0.20 | 3.8 (1.7-10) | 57 ± 16 |
| CBC | n.d. | 1.1 ± 0.16*** | n.d. | 0.53 ± 0.27 | >30 | 37 ± 13 |
| 7:1 | n.d. | 3.9 ± 2.6*** | >30 | −1.1 ± 0.82 | >30 | 27 ± 14 |
| 1:3 | n.d. | 5.9 ± 4.5*** | n.d. | 0.38 ± 0.46 | >30 | 35 ± 10 |

Cannabinoid modulation of catalepsy, body temperature, and nociception in C57BL/6 mice. Male and female mice were treated with 0 (vehicle, olive oil) or 0.3-30 mg/kg p.o. compounds. For 1:6.92 (CBC:THC) and 3.90:1 (CBC:THC) products, this dosing was based on the mg/kg dose of THC received. Graphs are presented in FIG. 1. Data are mean with 95% C.I. or ±S.E.M. Data were fit to a log [concentration] vs. response 3-parameter model in GraphPad Prism (v. 9.2). N=6-8 mice/treatment. ***p<0.001 compared to THC within test as determined by one-way ANOVA followed by Dunnett's post-hoc test.

TABLE 3
Assessment of mouse OFT responses following cannabinoid oral gavage.

| | Movement | | | Anxiety-reduction | |
|---|---|---|---|---|---|
| | ED₅₀ (95% C.I.) | | Eₘₐₓ ± S.E.M. | ED₅₀ (95% C.I.) | Eₘₐₓ ± S.E.M. |
| Compound | (mg/kg) | | (cm) | (mg/kg) | (sec) |
| THC | 6.6 | (0.66-10) | 1,700 ± 980 | >30 | 81 ± 72 |
| CBC | 35 | (17-100)* | 1,600 ± 340 | n.c. | 32 ± 7.2 |
| 7:1 | 28 | (9.4-210) | 1,100 ± 340 | >30 | 17 ± 7.8 |

| | | | | |
|---|---|---|---|---|
| 1:3 | >30 | 2,600 ± 620 | 2.6 | 16 ± 5.3 |

Cannabinoid modulation of movement in the OFT in C57BL/6 mice. Male and female mice were treated with 0 (vehicle, olive oil) or 0.3 −30 mg/kg p.o. compounds. For 1:6.92 (CBC:THC) and 3.90:1 (CBC:THC) products, this dosing was based on the mg/kg dose of THC received. Locomotion and time in the central quadrant of the OFT were made 30 minutes after compound administration. Data are mean with 95% C.I. or ±S.E.M. Data were fit to a log [concentration] vs. response 3-parameter model in GraphPad Prism (v. 9.2). N=6-8 mice/treatment. Corresponding graphs are presented in FIG. 2. *p<0.05 compared to THC within test as determined by one-way ANOVA followed by Dunnett's post-hoc test.

TABLE 4
Analyte: CBC

| | | | | | |
|---|---|---|---|---|---|
| | AUC | tₘₐₓ | Cₘₐₓ | t_{1/2} | F |
| Treatment | (ng*min/mL) | (min) | (ng/mL) | (h, min) | (%) |

THC i.v.

| | | | | | |
|---|---|---|---|---|---|
| Male | — | — | — | — | — |
| Female | — | — | — | — | — |
| Combined | — | — | — | — | — |

CBC i.v.

| | | | | | |
|---|---|---|---|---|---|
| Male | 131,939 ± 38,132 | 10 | 1,300 ± 500 | 1 h 42 min | 100 |
| Female | 114,900 ± 60,012 | 10 | 630 ± 160 | 3 h 8 min | 100 |
| Combined | 123,419 ± 51,094 | 10 | 960 ± 270 | 2 h 4 min | 100 |

7:1 i.v.

| | | | | | |
|---|---|---|---|---|---|
| Male | 1,349 ± 397 | 10 | 11 ± 2.4 | 2 h 52 min | 100 |
| Female | 1,357 ± 795 | 10 | 8.6 ± 4.1 | 3 h 27 min | 100 |
| Combined | 1,353 ± 568 | 10 | 9.7 ± 2.2 | 3 h 9 min | 100 |

1:3 i.v.

| | | | | | |
|---|---|---|---|---|---|
| Male | 31,845 ± 7,465 | 60 | 100 ± 24 | 9 h 50 min | 100 |
| Female | 30,295 ± 3,816 | 10 | 160 ± 30 | 3 h 30 min | 100 |
| Combined | 31,070 ± 6,627 | 10 | 130 ± 20 | 4 h 32 min | 100 |

THC p.o.

| | | | | | |
|---|---|---|---|---|---|
| Male | — | — | — | — | — |
| Female | — | — | — | — | — |
| Combined | — | — | — | — | — |

CBC p.o.

| | | | | | |
|---|---|---|---|---|---|
| Male | 35,444 ± 24,091 | 60 | 160 ± 64 | 5 h 36 min | 27 |
| Female | 61,220 ± 29,475 | 60 | 310 ± 79 | 5 h 25 min | 53 |
| Combined | 48,332 ± 28,054 | 60 | 235 ± 53 | 5 h 29 min | 39 |

7:1 p.o.

| | | | | | |
|---|---|---|---|---|---|
| Male | 2,471 ± 763 | 60 | 7.7 ± 1.1 | >12 h | 183 |
| Female | 3,780 ± 789 | 60 | 17 ± 1.4 | 8 h 56 min | 279 |
| Combined | 3,125 ± 1,052 | 60 | 12 ± 1.7 | >12 h | 231 |

1:3 p.o.

| | | | | | |
|---|---|---|---|---|---|
| Male | 115,697 ± 77,693 | 60 | 580 ± 210 | 9 h 9 min | 363 |
| Female | 119,110 ± 63,399 | 60 | 610 ± 170 | 6 h 37 min | 393 |
| Combined | 117,403 ± 63,491 | 60 | 590 ± 120 | 6 h 29 min | 378 |

Pharmacokinetic measurement of CBC in C57BL/6 mice. Male and female mice were treated with 10 mg/kg i.v. or p.o. of compounds. For 1:6.92 (CBC:THC) and 3.90:1 (CBC:THC) compositions, this dosing was based on the mg/kg dose of THC received. Blood samples were drawn 10 minutes, 1 hour, 6 hours, and 12 hours after compound administration. Data are mean±S.E.M. with individual data points. N=3-6 mice/treatment. Corresponding data are shown in FIG. 6.

TABLE 5
Analyte: THC

| | | | | | |
|---|---|---|---|---|---|
| | AUC | tₘₐₓ | Cₘₐₓ | t_{1/2} | F |
| Treatment | (ng*min/mL) | (min) | (ng/mL) | (h, min) | (%) |

THC i.v.

| | | | | | |
|---|---|---|---|---|---|
| Male | 197,430 ± 59,531 | 10 | 1,900 ± 1,000 | 3 h 9 min | 100 |
| Female | 121,624 ± 10,650 | 10 | 680 ± 2.0 | 7 h 59 min | 100 |
| Combined | 159,527 ± 43,826 | 10 | 1,300 ± 510 | 2 h 54 min | 100 |

CBC i.v.

| | | | | | |
|---|---|---|---|---|---|
| Male | — | — | — | — | — |
| Female | — | — | — | — | — |
| Combined | — | — | — | — | — |

7:1 i.v.

| | | | | | |
|---|---|---|---|---|---|
| Male | 9,509 ± 3,977 | 10 | 74 ± 40 | 2 h 59 min | 100 |
| Female | 8,015 ± 6,306 | 10 | 82 ± 80 | 1 h 46 min | 100 |
| Combined | 8,762 ± 4,865 | 10 | 78 ± 41 | 1 h 27 min | 100 |

1:3 i.v.

| | | | | | |
|---|---|---|---|---|---|
| Male | 10,555 ± 1,780 | 10 | 46 ± 18 | 3 h 59 min | 100 |
| Female | 13,089 ± 2,683 | 10 | 86 ± 28 | 3 h 52 min | 100 |
| Combined | 11,822 ± 2,589 | 10 | 66 ± 15 | 2 h 30 min | 100 |

THC p.o.

| | | | | | |
|---|---|---|---|---|---|
| Male | 7,297 ± 768 | 60 | 19 ± 0.67 | n.c. | 3.7 |
| Female | 40,333 ± 42,712 | 60 | 210 ± 0.64 | n.c. | 33 |
| Combined | 23,815 ± 29,881 | 60 | 120 ± 2.8 | 7 h 18 min | 15 |

CBC p.o.

| | | | | | |
|---|---|---|---|---|---|
| Male | — | — | — | — | — |
| Female | — | — | — | — | — |
| Combined | — | — | — | — | — |

7:1 p.o.

| | | | | | |
|---|---|---|---|---|---|
| Male | 15,671 ± 3,406 | 60 | 61 ± 0.92 | n.c. | 160 |
| Female | 24,710 ± 6,315 | 60 | 110 ± 0.71 | n.c. | 310 |
| Combined | 20,190 ± 6,107 | 60 | 86 ± 16 | 7 h 7 min | 230 |

1:3 p.o.

| | | | | | |
|---|---|---|---|---|---|
| Male | 34,963 ± 28,607 | 60 | 180 ± 22 | n.c. | 330 |
| Female | 23,639 ± 11,457 | 60 | 120 ± 1.0 | n.c. | 180 |
| Combined | 29,301 ± 19,964 | 60 | 150 ± 58 | 4 h 50 min | 250 |

Pharmacokinetic measurement of THC in C57BL/6 mice. Male and female mice were treated with 10 mg/kg i.v. or p.o. of compounds. For 1:6.92 (CBC:THC) and 3.90:1 (CBC:THC) compositions, this dosing was based on the mg/kg dose of THC received. Blood samples were drawn 10 minutes, 1 hour, 6 hours, and 12 hours after compound administration. Data are mean±S.E.M. with individual data points. N=3-6 mice/treatment. Corresponding data are shown in FIG. 6.

TABLE 6
Analyte: 11-OH-THC

| | | | | | |
|---|---|---|---|---|---|
| Treat- | AUC | tₘₐₓ | Cₘₐₓ | t_{1/2} | F_{Relative} |
| ment | (ng*min/mL) | (min) | (ng/mL) | (h, min) | (%) |

THC i.v.

| | | | | | |
|---|---|---|---|---|---|
| Male | 11,308 ± 5,844 | 10 | 85 ± 65 | 3 h 12 min | 100 |
| Female | 7,866 ± 1,965 | 60 | 26 ± 6.2 | 11 h | 100 |
| | | | | 43 min | |
| Combined | 9,627 ± 3,813 | 10 | 46 ± 32 | 4 h 37 min | 100 |

CBC i.v.

| | | | | | |
|---|---|---|---|---|---|
| Male | — | — | — | — | — |
| Female | — | — | — | — | — |
| Combined | — | — | — | — | — |

7:1 i.v.

| | | | | | |
|---|---|---|---|---|---|
| Male | 404 ± 92 | 10 | 1.6 ± 0.74 | 4 h 41 min | 100 |
| Female | 554 ± 442 | 10 | 4.2 ± 4.0 | 2 h 32 min | 100 |
| Combined | 479 ± 291 | 10 | 2.9 ± 2.1 | 3 h 10 min | 100 |

1:3 i.v.

| | | | | | |
|---|---|---|---|---|---|
| Male | 485 ± 170 | 10 | 1.7 ± 1.0 | 4 h 53 min | 100 |
| Female | 833 ± 267 | 10 | 4.0 ± 1.9 | 3 h 16 min | 100 |
| Combined | 659 ± 264 | 10 | 2.8 ± 1.2 | 3 h 40 min | 100 |

THC p.o.

| | | | | | |
|---|---|---|---|---|---|
| Male | 2,987 ± 160 | 60 | 6.4 ± 0.24 | n.c. | 26 |
| Female | 6,419 ± 5,883 | 60 | 27 ± 22 | 9 h 33 min | 82 |
| Combined | 4,703 ± 3864 | 60 | 17 ± 10 | >12 h | 49 |

CBC p.o.

| | | | | | |
|---|---|---|---|---|---|
| Male | — | — | — | — | — |
| Female | — | — | — | — | — |
| Combined | — | — | — | — | — |

7:1 p.o.

| | | | | | |
|---|---|---|---|---|---|
| Male | 2,523 ± 549 | 60 | 5.7 ± 0.81 | n.c. | 620 |
| Female | 3,716 ± 1,648 | 60 | 11 ± 1.5 | >12 h | 670 |
| Combined | 3,120 ± 1,202 | 60 | 8.3 ± 1.4 | >12 h | 650 |

1:3 p.o.

| | | | | | |
|---|---|---|---|---|---|
| Male | 2,221 ± 681 | 60 | 7.5 ± 2.3 | >12 h | 460 |
| Female | 2,465 ± 690 | 60 | 11 ± 2.6 | 7 h 13 min | 300 |
| Combined | 2,343 ± 680 | 60 | 9.2 ± 1.9 | 9 h 11 min | 360 |

Pharmacokinetic measurement of 11-OH-THC in C57BL/6 mice. Male and female mice were treated with 10 mg/kg i.v. or p.o. of compounds. For 1:6.92 (CBC:THC) and 3.90:1 (CBC:THC) products, this dosing was based on the mg/kg dose of THC received. Blood samples were drawn 10 minutes, 1 hour, 6 hours, and 12 hours after compound administration. Data are mean±S.E.M. with individual data points. N=3-6 mice/treatment. Corresponding data are shown in FIG. 7.

TABLE 7
Analyte: COOH-THC

| | | | | | |
|---|---|---|---|---|---|
| Treat- | AUC | tₘₐₓ | Cₘₐₓ | t_{1/2} | F_{Relative} |
| ment | (ng*min/mL) | (min) | (ng/mL) | (h, min) | (%) |

THC i.v.

| | | | | | |
|---|---|---|---|---|---|
| Male | 50,104 ± 20,290 | 60 | 140 ± 61 | >12 h | 100 |
| Female | 45,839 ± 6,940 | 60 | 110 ± 26 | n.c. | 100 |
| Combined | 47,972 ± 13,131 | 60 | 120 ± 28 | n.c. | 100 |

CBC i.v.

| | | | | | |
|---|---|---|---|---|---|
| Male | — | — | — | — | — |
| Female | — | — | — | — | — |
| Combined | — | — | — | — | — |

7:1 i.v.

| | | | | | |
|---|---|---|---|---|---|
| Male | 1,037 ± 598 | 10 | 3.3 ± 2.0 | 6 h 50 min | 100 |
| Female | 3,185 ± 2,432 | 60 | 9.1 ± 8.8 | 6 h 41 min | 100 |
| Combined | 2,111 ± 1,694 | 10 | 6.2 ± 2.2 | 6 h 43 min | 100 |

1:3 i.v.

| | | | | | |
|---|---|---|---|---|---|
| Male | 2,154 ± 857 | 60 | 5.4 ± 3.0 | >12 h | 100 |
| Female | 3,836 ± 1,001 | 60 | 13 ± 3.7 | 8 h 39 min | 100 |
| Combined | 2,995 ± 1,040 | 60 | 9.1 ± 6.1 | 10 h | 100 |
| | | | | 20 min | |

THC p.o.

| | | | | | |
|---|---|---|---|---|---|
| Male | 11,270 ± 1,058 | 60 | 23 ± 3.7 | n.c. | 22 |
| Female | 37,189 ± 1,296 | 60 | 169 ± 1.7 | >12 h | 81 |
| Combined | 19,961 ± 12,893 | 60 | 71 ± 35 | >12 h | 42 |

CBC p.o.

| | | | | | |
|---|---|---|---|---|---|
| Male | — | — | — | — | — |
| Female | — | — | — | — | — |
| Combined | — | — | — | — | — |

7:1 p.o.

| | | | | | |
|---|---|---|---|---|---|
| Male | 8,530 ± 510 | 60 | 17 ± 0.64 | n.c. | 820 |
| Female | 16,053 ± 3,849 | 60 | 46 ± 3.3 | >12 h | 500 |
| Combined | 12,292 ± 3,593 | 60 | 32 ± 4.6 | n.c. | 580 |

1:3 p.o.

| | | | | | |
|---|---|---|---|---|---|
| Male | 5,980 ± 1,416 | 60 | 15 ± 4.0 | n.c. | 280 |
| Female | 12,819 ± .548 | 60 | 43 ± 7.4 | >12 h | 330 |
| Combined | 9,399 ± 3,067 | 60 | 29 ± 2.7 | >12 h | 310 |

Pharmacokinetic measurement of COOH-THC in C57BL/6 mice. Male and female mice were treated with 10 mg/kg i.v. or p.o. of compounds. For 1:6.92 (CBC:THC) and 3.90:1 (CBC:THC) products, this dosing was based on the mg/kg dose of THC received. Blood samples were drawn 10 minutes, 1 hour, 6 hours, and 12 hours after compound administration. Data are mean±S.E.M. with individual data points. N=3-6 mice/treatment. Corresponding data are shown in FIG. 7.

Every document referenced herein, including publications and published patent documents, is hereby incorporated by reference herein in its entirety unless expressly excluded or otherwise limited. Reference to any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document will govern.

It is to be understood that the present disclosure, including description and drawings, are provided for the purpose of illustration and as an aid to understanding. The scope of the invention should not be limited in scope any embodiments or examples provided in the present disclosure, such as the application, details of construction or arrangements of the components. Except to the extent explicitly stated or inherent within the processes described, including any optional steps or components thereof, no required order, sequence, or combination is intended or implied. The invention is capable of other embodiments and of being practiced and carried out in various ways. Also, it is to be understood that the phraseology and terminology employed herein are for the purpose of description and should not be regarded as limiting. The scope of the claims should not be limited by the preferred embodiments set forth in the disclosure but should be given the broadest interpretation consistent with the disclosure as a whole.

1. A composition comprising a THC-type cannabinoid and a CBC-type cannabinoid.

2. The composition of claim 1, wherein when the composition is administered to a subject, induces a modulated high in the subject as compared to a control composition that does not contain the CBC-type cannabinoid.

3. The composition of claim 2, wherein the modulated high in the subject includes a modulated impairment, mood alteration, a combination thereof, as compared to the control composition.

4. The composition of claim 3, wherein the modulated impairment in the subject includes a modulated:
[240] impairment duration;
[241] sensory perception;
[242] executive function or cognition;
[243] memory; or
[244] any combination thereof,
as compared to a control composition that does not contain a CBC-type cannabinoid.

5. The composition of claim 3, wherein the modulated mood alteration in the subject includes a modulated:
[245] mood alteration duration;
[246] sociability;
[247] anger;
[248] confusion;
[249] depression;
[250] fatigue;
[251] despair;
[252] tension;
[253] vigor;
[254] grit;
[255] resiliency;
[256] sociability;
[257] willingness to explore; or
[258] any combination thereof,
as compared to a control composition without the CBC-type cannabinoid.

6. The composition of any one of claims 1 to 5, further comprising a CBC-type cannabinoid:THC-type cannabinoid ratio of from 100:1 to 1:100.

7. The composition of claim 6, wherein the CBC-type cannabinoid:THC-type cannabinoid ratio is from 10:1 to 1:10.

8. The composition of claim 7, wherein the CBC-type cannabinoid:THC-type cannabinoid ratio is about 4:1.

9. The composition of claim 8, wherein the CBC-type cannabinoid:THC-type cannabinoid ratio is about 1:7.

10. The composition of any one of claims 1 to 5, wherein the composition comprises from about 4% to about 99% of the CBC-type cannabinoid and from about 1% to about 96% of the THC-type cannabinoid by weight of total cannabinoids.

11. The composition of any one of claims 1 to 5, wherein the composition comprises from about 0.02 mg to about 5000 mg of the CBC-type cannabinoid and from about 0.5 mg to about 5000 mg by weight of the THC-type cannabinoid.

12. The composition of any one of claims 1 to 11, further comprising cannabicyclol (CBL), cannabicitran (CBT), sesqui-CBC, sesqui-CBG, cannabidiol (CBD), cannabigerol (CBG), or any combinations thereof.

13. The composition of any one of claims 1 to 12, wherein the THC-type cannabinoid comprises tetrahydrocannabinol (THC), tetrahydrocannabivarinol (THCV), tetrahydrocannabibutol (THCB), tetrahydrocannabihexol (THCH), tetrahydrocannabiphorol (THCP), hexahydrocannabinol (HHC), hexahydrocannabivarinol (HHCV), hexahydrocannabibutol (HHCB), hexahydrocannabihexol (HHCH), hexahydrocannabiphorol (HHCP), cannabinol (CBN), cannabivarinol (CBNV), cannabibutol (CBNB), cannabihexol (CBNH), cannabiphorol (CBNP), tetrahydrocannabinol-o-acetate (THC-O-Ac), tetrahydrocannabivarinol-o-acetate (THCV-O-Ac), tetrahydrocannabibutol-o-acetate (THCB-O-Ac), tetrahydrocannabihexol-o-acetate (THCH-O-Ac), tetrahydrocannabiphorol-o-acetate (THCP-O-Ac), 11-hydroxy-tetrahydrocannabinol (11-OH-THC), 11-hydroxy-tetrahydrocannabivarinol (11-OH-THCV), 11-hydroxy-tetrahydrocannabibutol (11-OH-THCB), 11-hydroxy-tetrahydrocannabihexol (11-OH-THCH), 11-hydroxy-tetrahydrocannabiphorol (11-OH-THCP), or a combination thereof.

14. The composition of claim 13, wherein the THC-type cannabinoid comprises THC.

15. The composition of claim 14, wherein the THC comprises delta-8 THC, delta-9 THC, or both.

16. The composition of claim 15, wherein the THC comprises delta-9 THC.

17. The composition of claim 16, wherein the CBC-type cannabinoid comprises cannabichromevarin (CBCV), cannabichromebutol (CBCB), cannabichromene (CBC), cannabichromehexol (CBCH), cannabichromephorol (CBCP), cannabichromebutol (CBCB), cannabichromehexol (CBCH), sesqui-CBC, sesqui-CBCV, sesqui-CBCB, sesqui-CBCH, sesqui-CBCP, or a combination thereof.

18. The composition of claim 17, wherein the CBC-type cannabinoid comprises CBC.

19. The composition of any one of claims 1 to 17, wherein the composition is for oral administration.

20. The composition of claim 19, further comprising a taste masking agent.

21. The composition of any one of claims 1 to 18, wherein the composition is for pulmonary administration.

22. The composition of any of claims 1 to 18, wherein the composition is a dosage form.

23. The composition of claim 23, wherein the dosage form is a tablet, a liquid, a capsule, a film, a powder, an aerosol, a gel, a cream, an ointment, or any combination thereof.

24. The composition of claim 24, wherein the dosage form further comprises at least one pharmaceutically acceptable carrier.

25. The composition of any of claim 23 or 24, wherein the dosage form is a tablet, liquid capsule, or film and further comprises a masking agent.

26. A composition for use with a THC-type cannabinoid, wherein the composition comprises a CBC-type cannabinoid.

27. The composition of claim 26, wherein when the THC-type cannabinoid and composition are administered to a subject, the THC-type cannabinoid induces a high in the subject, and the composition modulates the high.

28. The composition of claim 27, wherein the high includes impairment, mood alteration, or both in the subject, and the composition modulates the impairment, mood alteration, or both in the subject

29. The composition of claim 28, wherein the modulation of the impairment of the subject comprises modulation of:
[259] impairment duration;
[260] sensory perception;
[261] executive function or cognition;
[262] memory; or
[263] any combination thereof.

30. The composition of claim 28, wherein the modulation of the mood alteration of the subject comprises modulation of:
[264] mood alteration duration;
[265] sociability;
[266] anger;
[267] confusion;
[268] depression;
[269] fatigue;
[270] despair;
[271] tension;
[272] vigor;
[273] grit;
[274] resiliency;
[275] sociability;
[276] willingness to explore; or
[277] any combination thereof.

31. The composition of any one of claims 26 to 30, wherein the CBC-type cannabinoid is adapted to be included with the THC-type cannabinoid in a CBC: THC-type cannabinoid ratio of from 100:1 to 1:100.

32. The composition of claim 31, wherein the CBC-type cannabinoid:THC-type cannabinoid ratio is from 10:1 to 1:10.

33. The composition of claim 32, wherein the CBC-type cannabinoid:THC-type cannabinoid ratio is about 4:1.

34. The composition of claim 32, wherein the CBC-type cannabinoid:THC-type cannabinoid ratio is about 1:7.

35. The composition of any one of claims 26 to 34, further comprising cannabicyclol (CBL), cannabicitran (CBT), sesqui-CBC, sesqui-CBG, cannabidiol (CBD), cannabigerol (CBG), or any combinations thereof.

36. The composition of any one of claims 26 to 35, wherein the THC-type cannabinoid comprises tetrahydrocannabinol (THC), tetrahydrocannabivarinol (THCV), tetrahydrocannabibutol (THCB), tetrahydrocannabihexol (THCH), tetrahydrocannabiphorol (THCP), hexahydrocannabinol (HHC), hexahydrocannabivarinol (HHCV), hexahydrocannabibutol (HHCB), hexahydrocannabihexol (HHCH), hexahydrocannabiphorol (HHCP), cannabinol (CBN), cannabivarinol (CBNV), cannabibutol (CBNB), cannabihexol (CBNH), cannabiphorol (CBNP), tetrahydrocannabinol-o-acetate (THC-O-Ac), tetrahydrocannabivarinol-o-acetate (THCV-O-Ac), tetrahydrocannabibutol-o-acetate (THCB-O-Ac), tetrahydrocannabihexol-o-acetate (THCH-O-Ac), tetrahydrocannabiphorol-o-acetate (THCP-O-Ac), 11-hydroxy-tetrahydrocannabinol (11-OH-THC), 11-hydroxy-tetrahydrocannabivarinol (11-OH-THCV), 11-hydroxy-tetrahydrocannabibutol (11-OH-THCB), 11-hydroxy-tetrahydrocannabihexol (11-OH-THCH), 11-hydroxy-tetrahydrocannabiphorol (11-OH-THCP), or any combinations thereof.

37. The composition of claim 36, wherein the THC-type cannabinoid comprises THC.

38. The composition of claim 37, wherein the THC comprises delta-8 THC, delta-9 THC, or both.

39. The composition of claim 38, wherein the THC comprises delta-9 THC.

40. The composition of any one of claims 26 to 39, wherein the CBC-type cannabinoid comprises cannabichromevarin (CBCV), cannabichromebutol (CBCB), cannabichromene (CBC), cannabichromehexol (CBCH), cannabichromephorol (CBCP), cannabichromebutol (CBCB), cannabichromehexol (CBCH), sesqui-CBC, sesqui-CBCV, sesqui-CBCB, sesqui-CBCH, sesqui-CBCP, or any combination thereof.

41. The composition of claim 40, wherein the CBC-type cannabinoid comprises CBC.

42. The composition of any one of claims 26 to 41, wherein the composition is for oral administration.

43. The composition of any one of claims 26 to 41, wherein the composition is for pulmonary administration.

44. A use of the composition of any one of claims 26 to 43, to modulate the effects of a THC-type cannabinoid.

45. A use of a composition of any one of claims 1 to 25, to deliver a modulated effect of a THC-type cannabinoid.

46. A product comprising the composition of any of claims 1 to 21.

47. The product of claim 46, wherein the product comprises an inhalable cannabis product;
an ingestible cannabis product; a topical cannabis product; or any combination thereof.

48. The product of claim 46 or 47, wherein the product comprises an inhalable cannabis product, an ingestible cannabis product, or any combination thereof.

49. The product of claim 48, wherein the product comprises an inhalable cannabis product.

50. The product of claim 49, wherein the product is a vaporizer, vaporizer cartridge, vaporizer composition, joint, blunt, or spliff.

51. The product of claim 48, wherein the product is an ingestible cannabis product.

52. The product of claim 51, wherein the product is a cannabis edible, beverage, or a tincture.

53. The product of claim 52, wherein the cannabis edible is a gummy, chocolate, confection or baked good.

54. The product of claim 53, wherein the cannabis edible is a gummy.

55. A method of modulating the effect of a THC-type cannabinoid to be administered to a subject comprising:
administering a THC-type cannabinoid to the subject, and
administering the composition of any one of claims 26 to 43 to the subject, wherein the THC-type cannabinoid and the CBC-type cannabinoid can be administered separately to the subject or in a single composition.

56. A method of delivering a modulated effect of a THC-type cannabinoid comprising:
administering to a subject the composition of any one of claims 1 to 25.
